(19) United States
(12) Patent Application Publication
(10) Pub. No.: US 2016/0067273 A1
(43) Pub. Date: Mar. 10, 2016

(54) Compositions and methods for preparation of synthetic alpha-gal nanoparticles and for their clinical use

(71) Applicant: Uri Galili, Chicago, IL (US)

(72) Inventor: Uri Galili, Chicago, IL (US)

(21) Appl. No.: 14/756,138

(22) Filed: Aug. 5, 2015

Related U.S. Application Data
(60) Provisional application No. 62/070,979, filed on Sep. 10, 2014.

(51) Int. Cl. A61K 31/715; A61L 27/36; A61L 27/16; A61L 27/54

(52) U.S. Cl. CPC A61K 31/715; A61L 27/54; A61L 27/3612; A61L 27/16; A61L 2300/232; A61L 2300/412

(57) ABSTRACT

The present invention is related to the field of healing of internal injuries. In particular, the present invention provides compositions and methods comprising molecules and nanoparticles with linked α-gal epitopes from synthetic origin for induction of recruitment and activation of macrophages within or surrounding injured tissue of treated patients. These macrophages further recruit stem cells into the injured tissues. The recruited macrophages and stem cells promote the repair and regeneration of the treated injured tissue. This invention further teaches methods and compositions comprising molecules and nanoparticles with linked α-gal epitopes of synthetic origin for inducing recruitment and activation of macrophages into biomaterial implants for improving the conversion of such implants into functional tissues and organs within treated patients.

### CROSS REFERENCE TO RELATED APPLICATIONS

This application claims the benefit of U.S. provisional patent application 62/070,979 filed on Sep. 10, 2014, the contents of which are incorporated in their entirety.

### FIELD OF INVENTION

The present invention is related to the fields of wound healing, regeneration and remodeling of injured tissues and implantation of biomaterials, also called bio-implants. This invention provides compositions and methods comprising synthetic nanoparticles with linked α-gal epitopes for induction of rapid recruitment and activation of macrophages that mediate accelerated healing and regeneration of external and internal injured tissues by secreting a variety of cytokines and growth factors which induce healing of injuries, recruit stem cells and generate microenvironments that support stem cell differentiation in injuries and in bio-implants.

### BACKGROUND OF THE INVENTION

The present invention teaches how to prepare synthetic α-gal nanoparticles and how to use synthetic α-gal nanoparticles for inducing recruitment and activation of macrophages in injured external and internal tissues. The α-gal nanoparticles are biodegradable nanoparticles expressing multiple α-gal epitopes and their size can range between 1.0 nanometer (nm) to 0.5 millimeter (mm), and preferably 10-200 nm. It is contemplated that this recruitment and activation of macrophages accelerates physiologic processes that mediate repair and regeneration of the treated injured tissues such as, but not limited to wounds and burns, pilonidals, ischemic heart muscle, skeletal muscle, injured nerves, incisions, injured bones and injured cartilage. The present invention further teaches the use of synthetic α-gal nanoparticles in improving the efficacy of biomaterials used for tissue engineering such as, but not limited to, collagen sheets, decellularized tissues and decellularized organs. Upon implantation of such biomaterials containing synthetic α-gal nanoparticles in patients these nanoparticles will induce rapid recruitment of macrophages into the implant and induce activation of these recruited macrophages. It is contemplated that this recruitment and activation of macrophages will initiate physiologic processes inducing effective regeneration of the implant which ultimately will convert it into biologically functioning tissue or organ. The present invention also describes the use of natural or synthetic α-gal nanoparticles for activating hair follicles in order to induce hair growth on the head or other body parts.

Macrophages are the pivotal cells in early stages of injury healing and tissue regeneration. Macrophages migrating into injury sites debride the injured tissue by phagocytosis. Macrophages debride the tissue of dead cells. Subsequently, upon transition into the pro-healing phase, macrophages orchestrate regeneration by secreting a variety of cytokines/growth factors that induce that facilitate the repair and regeneration processes in the injured tissue and regeneration of the injured tissue (Bryant et al., Prog Clin Biol Res, 266:273, 1988; Knighton and Fiegel, Prog Clin Biol Res, 299:217, 1989; DiPietro, Shock 4:233, 1995). Macrophages also secrete cytokines and growth factors that are essential in further recruitment of macrophages, as well as recruitment of stem cells into the injury site (Lolmede et al. J Leukoc Biol 85: 779, 2009). Cytokines and growth factors also regulate fibroblast and epithelial cell proliferation, as well as proliferation of endothelial cells for revascularization of the injured tissue (Rappolee and Werb, Curr Top Microbiol Immunol, 181:87, 1992) Compositions and methods for inducing rapid recruitment of macrophages into injured tissues, thereby accelerate the repair and regeneration of these tissues are desirable for decreasing morbidity and for achieving a more effective repair of injured tissues than the post injury physiologic repair. This is because in many cases the slow pace of physiologic repair results in fibrosis and formation of a scar tissue rather than in restoration of the original structure and function of the injured tissue. The use of synthetic α-gal nanoparticles enables a fast enough repair and regeneration process that avoids fibrosis and scar formation and enables restoration of structure and function of the treated tissue.

Macrophages are physiologically recruited into wounds within several days by cytokines such as MIP-1, MCP-1 and RANTES released from cells within and around injury sites (Low et al. Am J Pathol 159: 457, 2001; Heinrich et al. 11: 110, 2003; Shukaliak and Dorovini-Zis J Neuropathol Exp Neurol 59: 339, 2000). This recruitment of macrophages into skin wounds and internal injured tissues can be markedly accelerated by antibodies interacting with various antigens and causing local activation of the complement system. Complement activation results in generation of complement cleavage peptides such as C5a, C4a and C3a which are chemotactic factors that induce rapid extravasation of monocytes, and their differentiation into macrophages which migrate along the chemotactic gradient (Snyderman and Pike Annu Rev Immunol 2:257, 1984).

Some studies indicated that macrophages also include populations of multipotential stem cells (Seta and Kuwana, Keio J. Med 56: 41, 2007). Other studies demonstrated recruitment of stem cells by macrophages that reach the injury sites (Lolmede et al. supra). Whether stem cells are recruited by macrophages or originate from macrophages upon reaching the injured area, they receive instructive cues from differentiated cells located nearby, from the extracellular matrix (ECM) and from the microenvironment, which direct the stem cells to differentiate into functional cells that repair and regenerate the injured tissue (Seta and Kuwana, supra; Stappenbeck and Miyoshi, Science, 324: 1666, 2009). If stem cells do not reach the injury site early enough after the injury, then the default repair mechanism which takes place is fibrosis mediated by fibroblasts that infiltrate the injury site. This fibrosis results in scar formation that is an irreversible process preventing restoration of the original tissue structure and function. Therefore, accelerated and improved repair and regeneration of damaged tissues is contemplated to be achievable by therapeutic induction for recruitment of monocytes into the injured tissue and induction of these cells to mature into macrophages that promote tissue repair. Such macrophages, in turn, secrete a variety of cytokines and growth factors that promote angiogenesis, as well as induce recruitment and activation of stem cells that repopulate the injured tissue and regenerate it for restoration of the pre-injury biological activity (Stein and Keshav, Clin Exp Allergy 22:19, 1992; DiPietro, supra; Clark, J Dermatol Surg Oncol, 19:693, 1993; and Rappolee and Werb, supra; Allison and Islam J. Pathol 217: 144, 2009).

Previous inventions (U.S. Pat. No. 8,084,057, U.S. Pat. No. 8,440,198 and U.S. Pat. No. 8,865,178) have been teaching how to accelerate and improve healing and regeneration within injured external and internal human tissues by harnessing the immunological potential of the natural anti-Gal antibody. The harnessing of this antibody is feasible by the use of α-gal nanoparticles. These inventions demonstrated the use of natural materials such as rabbit red cell α-gal glycolipids, phospholipids and cholesterol for preparation of submicroscopic liposomes, called α-gal nanoparticles, which express an abundance of α-gal epitopes. The α-gal nanoparticles bind effectively the natural anti-Gal antibody which is the most abundant natural antibody in humans and which interacts specifically with α-gal epitopes. These three inventions further teach the use of these α-gal nanoparticles for rapid recruitment and activation of macrophages in order to accelerate healing of wounds and burns as well as inducing healing and regeneration of internal injuries such as, but not limited to post infarct ischemic myocardium, injured nerve, damaged orthopedic tissues such as cartilage and bone. In addition, patent application #20140220052 filed on Mar. 26, 2014 teaches the use of α-gal nanoparticles for induction and remodeling of bio-implants from mammalian origin.

The present invention teaches compositions and methods for preparation of synthetic α-gal nanoparticles that can replace the α-gal nanoparticles generated from natural materials in the induction of repair and regeneration of injuries as well as for the effective remodeling and regeneration of bio-implants of mammalian origin and for induction of hair growth by activation of quiescent hair follicles.

This invention describes how injection or application of synthetic α-gal nanoparticles by various methods into ischemic myocardium post infarction induces rapid recruitment of macrophages to the injection site and local activation of the recruited macrophages. These activated macrophages may further recruit stem cells that will be guided by preserved dead cardiomyocytes and the preserved extracellular matrix (ECM) to differentiate into functional cardiomyocytes, thereby restoring the biological activity of the injured myocardium. In addition, the invention describes injection or application of synthetic α-gal nanoparticles by various methods to nerve injuries results in a recruitment and activation of macrophages in nerve injury sites and induction of local angiogenesis which is a prerequisite for effective axonal sprouting in order to bridge the neural lesion area, grow into the post lesion (distal) axonal tube and to regenerate the nerve. In the absence of rapid and extensive recruitment and activation of macrophages, the default mechanism of fibrosis of the ischemic myocardium or the injured nerve will result in permanent prevention of regeneration of the injured site. A similar injection or application of α-gal nanoparticles to other internal injuries will induce the rapid recruitment and activation of macrophages which ultimately will accelerate and improve the efficacy of the repair and regeneration mechanism for restoring the biological activity of the treated injured tissues.

This invention further describes methods for the incorporation of α-gal nanoparticles into biomaterials which are used in tissue engineering and which will increase the efficacy of such biomaterials in regeneration of injured tissues. The synthetic α-gal nanoparticles placed within biomaterials such as, but not limited to, collagen sheets and decellularized tissues and organs will induce rapid and extensive recruitment of macrophages upon implantation of such biomaterials. The synthetic α-gal nanoparticles will further induce activation of these recruited macrophages which, in turn, will produce cytokines/growth factors that will induce effective recruitment of stem cells into the implanted biomaterials. When natural decellularized tissues and organs containing synthetic α-gal nanoparticles are used for implantation, the stem cells recruited into these implants will receive guidance from the microenvironment and ECM scaffold for the differentiation into cells that repopulate the biomaterial and restore biological activity of the damaged tissue or organ replaced by the biomaterial.

### SUMMARY OF THE INVENTION

This invention teaches methods for preparing synthetic α-gal nanoparticles and using them in order to induce rapid recruitment of macrophages into internal tissues that are injured and induce activation of these macrophages to produce cytokines and growth factors that facilitate recruitment of stem cells and induce repair and regeneration of the injured tissue. This invention further teaches the application of synthetic α-gal nanoparticles into biomaterials including, but not limited to decellularized tissues and organs, in order to induce rapid recruitment of macrophages into biomaterials once they are implanted and in order to induce activation of the recruited macrophages. These recruited and activated macrophages produce cytokines and growth factors that recruit stem cells and improve the efficacy and pace of regeneration of biomaterials implanted for the purpose of tissue engineering. α-Gal nanoparticles refer to biodegradable liposomes of submicroscopic size as well as liposomes of microscopic size which present multiple α-gal epitopes. The α-gal epitope is a carbohydrate antigen with the structure Galα1-3Galβ1-4GlcNAc-R or other carbohydrate epitopes carrying terminal α-galactosyl residues that binds the human natural anti-Gal antibody. Anti-Gal is the most abundant natural antibody in all humans, constituting ˜1% of immunoglobulins. Anti-Gal binding to α-gal nanoparticles that are introduced into injured tissues, or binding to α-gal nanoparticles within implanted biomaterials, activates the complement system thereby generating chemotactic complement cleavage peptides that induce rapid and extensive recruitment of macrophages. The subsequent interaction between Fc portion of anti-Gal coating α-gal nanoparticles and Fcγ receptors on macrophages activates these cells to produce cytokines and growth factors (referred to as cytokines/growth factors) that promote injury repair and recruit stem cells and repopulate the tissue and the implant with cells that restore the biological activity of the tissue or regenerate the implanted decellularized tissue or organ. Previous inventions (U.S. Pat. No. 8,084,057, U.S. Pat. No. 8,440,198 and U.S. Pat. No. 8,865,178) and patent application #20140220052 demonstrated the use of α-gal nanoparticles prepared from rabbit red cell membranes. Membranes of rabbit red cells provide phospholipids and cholesterol which are found also in human red cell membranes, as well as high amounts of glycolipids carrying the α-gal epitope, called α-gal glycolipids. Extraction of these phospholipids, cholesterol and α-gal glycolipids, followed by their drying and sonication in saline results in the generation of α-gal nanoparticles.

Since preparation of nanoparticles from materials that do not originate in mammals is preferable for a number of pharmaceutical companies and because of the need to provide to some regulatory agencies exact definition of content of drugs, it is of interest to develop compositions and method for generating synthetic α-gal nanoparticles which are comprised of a well-defined phospholipid and a synthetic α-gal glycolipid with known carbohydrate structures. The synthetic α-gal glycolipid molecules in synthetic α-gal nanoparticle bind the anti-Gal antibody upon application of the nanoparticles to wounds or into various sites in the body. The phospholipid molecules in synthetic α-gal nanoparticles stabilize the α-gal glycolipid molecules in the synthetic α-gal nanoparticles and thus prevent the insertion of α-gal glycolipids into cell membranes. This, in turn, prevents a subsequent binding of the natural anti-Gal antibody to normal cells in order to avoid destruction of the cells. A non-limiting example for preparation of synthetic α-gal nanoparticles is a mixture of phosphatidyl choline and a synthetic α-gal glycolipid having one or two or several α-gal epitopes linked by a spacer containing one or two or several hydrocarbons or hydrocarbons and other spacer components to a diacyl lipid or to monoacyl lipid. A non-limiting example of a synthetic α-gal glycolipid is FSL-Galili(tri) produced by KODE Biothech (Auckland, NZ) and sold by Sigma-Aldrich (St. Louis, Mo., USA) as catalogue number F9432 in Sigma catalogue. Mixing phosphatidyl choline with an α-gal glycolipid such as, but not limited to FSL-Galili(tri), drying of the mixture then sonicating the mixture in water, or saline solution, or any other hydrophilic solvent will result in formation of α-gal nanoparticles comprised of the α-gal glycolipid and phosphatidyl choline. The size of these nanoparticles will be inversely proportional to the intensity and length of the sonication process. The synthetic α-gal nanoparticles are brought to a size that enables their sterilization by filtration through a filter with pore size of 0.2 μm or by any other means of sterilization known to those skilled in the art.

In one embodiment, synthetic α-gal nanoparticles injected into ischemic myocardium induce extensive recruitment of macrophages that are activated to secrete cytokines/growth factors that preserving the structure of the ischemic tissue. These cytokines/growth factors further recruit stem cells into the ischemic myocardium are guided by the microenvironment and the ECM to differentiate into cardiomyocytes that repopulate the ischemic myocardium and restore its biological activity.

In another embodiment the synthetic α-gal nanoparticles are applied to nerve injury sites. These synthetic α-gal nanoparticles recruit macrophages into the nerve injury site. The recruited macrophages secrete cytokines/growth factors including, but not limited to vascular endothelial growth factor (VEGF) that induce local angiogenesis. This angiogenesis promotes axonal sprouting will secrete cytokines that recruit stem cells and promote axonal sprouting in order to bridge the neural lesion area, grow into the post lesion axonal tube and to regenerate the nerve. A similar injection or application of synthetic α-gal nanoparticles in other internal injuries will induce the rapid recruitment and activation of macrophages which ultimately may accelerate and improve the efficacy of the repair and regeneration mechanism for restoring the biological activity of the treated injured tissues. In the absence of rapid and extensive recruitment and activation of macrophages, the default mechanism of fibrosis will occur in the injured tissues and thus, will result in permanent prevention of regeneration of the injured site for restoration of the original biological activity.

This invention further teaches methods for the incorporation of synthetic α-gal nanoparticles into biomaterials which are used in tissue engineering and which will increase the efficacy of such biomaterials in regeneration of injured tissues. The synthetic α-gal nanoparticles are placed within biomaterials such as, but not limited to, collagen sheets and decellularized tissues and organs such as decellularized liver, heart, kidney, intestine, stomach, muscle, esophagus, cartilage, bone or any other decellularized organ or tissue. Implantation of biomaterials or decellularized tissues or organs containing synthetic α-gal nanoparticles into patients will result in binding of the natural anti-Gal antibody of the treated patient to the synthetic α-gal epitopes on these nanoparticles. This will result in activation of the complement system and thus generation of complement cleavage chemotactic peptides that induce rapid and extensive recruitment of macrophages into the implant. The synthetic α-gal nanoparticles will further induce activation of these recruited macrophages by the interaction between anti-Gal antibody molecules bound to these nanoparticles and Fcγ receptors on the macrophages. These activated macrophages will produce cytokines/growth factors that will induce effective recruitment of stem cells into the implanted biomaterials. When natural decellularized tissues and organs containing synthetic α-gal nanoparticles are used for implantation, or when biomaterials containing ECM are used as implants, the stem cells recruited into these implants will receive guidance from the microenvironment and ECM scaffold for the differentiation into cells that repopulate the biomaterial and restore biological activity of the damaged tissue or organ replaced by the biomaterial.

### BRIEF DESCRIPTION OF THE DRAWINGS

The following are illustrations of the present invention and are not intended as limiting in any manner.

FIG. 1 presents a schematic illustration of a synthetic α-gal nanoparticle comprised of phospholipids (round hydrophilic head facing outwards and elongated hydrophobic portion of fatty acid “tails” facing inward the particle) and synthetic α-gal glycolipids capped with an α-gal epitope. The phospholipids may form a monolayer as in a micelle presented in this figure, or bilayer as in a liposome. The synthetic α-gal glycolipid may have one or more than one branch(s) carrying α-gal epitopes. When synthetic α-gal nanoparticles are applied into an injured tissue, or when a biomaterial such as, but not limited to, a natural tissue or organ, containing synthetic α-gal nanoparticles are implanted in humans, the natural anti-Gal antibody binds to the α-gal epitopes on the synthetic nanoparticles. The following processes occur as a result of this antigen/antibody interaction and are numbered: 1. In vivo binding of the natural anti-Gal antibody to administered synthetic α-gal nanoparticles activates the complement system. 2. The chemotactic factors C5a and C3a generated as complement cleavage peptides induce rapid recruitment of macrophages to the site of synthetic α-gal nanoparticles. 3. The recruited macrophages interact via their Fcγ receptors (FcγR) with the Fc portion of anti-Gal coating the synthetic α-gal nanoparticles. 4. This interaction activates the macrophages to secrete a wide range of cytokines and growth factors that promote regeneration and recruit stem cells into the injury site. 5. The recruited stem cells receive cues from the extracellular matrix (ECM) to differentiate into mature cells that restore the original structure and function of the injured tissue.

FIG. 2. Demonstration of the interaction of human natural anti-Gal antibody with synthetic α-gal epitopes linked to bovine serum albumin (α-gal-BSA). Anti-Gal activity in the serum of 10 representative individuals from a healthy population, as assayed by ELISA with synthetic α-gal epitopes linked to bovine serum albumin (BSA) as solid phase antigen. Goat anti-human IgG antibody coupled to peroxidase was used as secondary antibody and Ortho-nitrophenyl diamine used as color substrate. Color reaction was stopped after 5 min with 1.0M sulfuric acid and color absorbance measured at 492 nm.

FIG. 3 describes a non-limiting example for the preparation of synthetic α-gal nanoparticles. The phospholipid choline is dissolved in an organic solvent such as, but not limited to, methanol. A synthetic α-gal glycolipids, such as, but not limited to pentasaccharide linked to a diacyl lipid is dissolved together with the phosphatidyl choline in methanol at a molar ratio such as, but not limited to 1:10 α-gal glycolipid:phospholipid. The mixture is dried in a rotary evaporator, or in any other drying device known to those skilled in the art. Subsequently, the dried mixture is sonicated to form synthetic α-gal nanoparticles comprised of phosphatidyl choline and α-gal glycolipid molecules. Synthetic α-gal nanoparticles may be prepared from any type of lipid, preferably from a phospholipid and from synthetic glycolipids comprised of one or more carbohydrate chains all or part of which carry α-gal epitopes. The α-gal epitopes may be linked to the lipid by a spacer or directly by a carbohydrate chain. This linking is performed by methods known to those skilled in the art.

### DEFINITIONS

To facilitate an understanding of the present invention, a number of terms and phrases are defined below:

The term “lipid” as used herein, refers to any molecule from a group of naturally occurring or synthetic molecules that include fats, waxes, sterols, fat-soluble vitamins, monoglycerides, diglycerides, triglycerides, phospholipids,

The term “α-gal epitope” as used herein, refers to any molecule or part of a molecule, with a terminal structure comprising Galα1-3Galβ1-4GlcNAc-R, Galα1-3Galβ1-3GlcNAc-R, or any carbohydrate chain with terminal Galα1-3Gal at the non-reducing end, or any molecule with terminal α-galactosyl unit capable of binding the anti-Gal antibody. The α-gal epitope may be of natural source or of synthetic source.

The term “glycolipid” as used herein, refers to any molecule with at least one carbohydrate chain linked to a ceramide, or a fatty acid chain, or any other lipid. Alternatively, a glycolipid may be referred to as a glycosphingolipid. Glycolipids may be of natural or synthetic origin.

The term “α-gal glycolipid” as used herein, refers to any glycolipid that has at least on “α-gal epitope on its non-reducing end of the carbohydrate chain and may be of natural or synthetic origin.

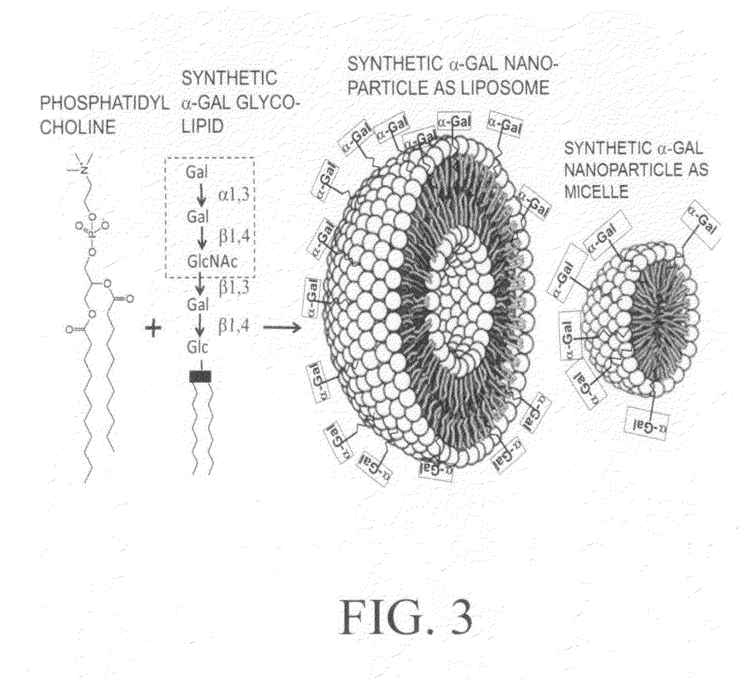

The term “α-gal liposomes” as used herein, refers to any liposomes comprised of natural or synthetic phospholipids, or other lipids, which is also comprised of hydrocarbon base, or any other base which contains α-gal epitopes or of α-gal epitopes in natural or synthetic α-gal glycolipids, α-gal proteins, α-gal proteoglycans, or α-gal polymers and which may or may not be comprised also of cholesterol. The liposome can be of any size provided that it has one or more lipid bilayer. The term α-gal nanoparticles also represents small α-gal liposomes Micelle is a spherical structure comprising lipids, including but not limited to phospholipids and glycolipids in which the hydrophobic tails are facing each other within the inner space of the sphere and the hydrophilic part faces the aqueous surrounding, as depict in FIG. 3.

The term “α-gal nanoparticles” as used herein, refers to nanoparticles comprised of natural or synthetic materials and which present natural or synthetic α-gal epitopes. α-gal epitopes may be part of α-gal glycolipids, α-gal glycoproteins, α-gal proteoglycans, synthetic α-gal comprised molecules or α-gal polymers. In this application, the example used for such nanoparticles are synthetic α-gal nanoparticles comprised of phospholipids and α-gal glycolipids. The term “α-gal nanoparticles” also includes α-gal liposomes of microscopic and submicroscopic size, ranging in size from 0.01 nm to 500 μm.

The term “synthetic α-gal nanoparticles” as used herein, refers to nanoparticles comprised of or natural or synthetic lipids, such as but not limited to phosphatidyl choline, phosphatidyl ethanolamine, phosphatidyl serine and synthetic α-gal glycolipids or any other synthetic or natural molecules that bind the natural anti-Gal antibody. The term “synthetic α-gal nanoparticles” also includes synthetic α-gal liposomes of microscopic and submicroscopic size, ranging in size from 1 nm to 500 μm and which are comprised of or natural or synthetic lipids, such as but not limited to phosphatidyl choline, phosphatidyl ethanolamine, phosphatidyl serine and synthetic α-gal glycolipids or any other synthetic or natural molecules that bind the natural anti-Gal antibody.

As used herein, the term “purified” refers to molecules (polynucleotides, or polypeptides, or glycolipids) that are removed from their natural environment, isolated or separated. “Substantially purified” molecules are at least 50% free, preferably at least 75% free, more preferably at least 90% and most preferably at least 95% free from other components with which they are naturally associated.

The terms “α1,3-galactosyltransferase,” “α-1,3-galactosyltransferase,” “α1,3GT,” “glycoprotein α-galactosyltransferase 1” and “GGTA1,” as used herein refer to any enzyme capable of synthesizing α-gal epitopes. The enzyme is expressed in nonprimate mammals but not in humans, apes and Old World monkeys. The carbohydrate structure produced by the enzyme is immunogenic in man and most healthy people have high titer natural anti α-gal antibodies, also referred to as “anti-Gal” antibodies. In some embodiments, the term “α1,3GT” refers to a common marmoset gene (e.g., Callithrix jacchus—GENBANK Accession No. S71333) and its gene product, as well as its functional mammalian counterparts (e.g., other New World monkeys, prosimians and non-primate mammals, but not Old World monkeys, apes and humans). In other embodiments, the term “α1,3GT” refers to mouse α1,3GT (e.g., Mus musculus—nucleotides 445 to 1560 of GENBANK Accession No. NM_{—}010283), bovine α1,3GT (e.g., Bos taurus—GENBANK Accession No. NM_{—}177511), feline α1,3GT (e.g., Felis catus—GENBANK Accession No. NM_{—}001009308), ovine α1,3GT (e.g., Ovis aries—GENBANK Accession No. NM_{—}001009764), rat α1,3GT (e.g., Rattus norvegicus—GENBANK Accession No. NM_{—}145674) and porcine α1,3GT (e.g., Sus scrofa—GENBANK Accession No. NM_{—}213810).

The term “anti-Gal binding epitope”, as used herein, refers to any molecule or part of molecule that is capable of binding in vivo the natural anti-Gal antibody.

The term “anti-Gal antibody”, as used herein, refers to a natural antibody present in large amounts in humans or in other vertebrate lacking α-gal epitopes and which binds to antigens carrying α-gal epitopes, molecules and peptides mimetic to α-gal epitopes and other carbohydrates that mimic α-gal epitopes structure or are part of this structure.

The term “isolated” as used herein, refers to any composition or mixture that has undergone a laboratory purification procedure including, but not limited to, extraction, centrifugation and chromatographic separation (e.g., thin layer chromatography or high performance liquid chromatography). Usually such purification procedures provide an isolated composition or mixture based upon physical, chemical, or electrical potential properties. Depending upon the choice of procedure an isolated composition or mixture may contain other compositions, compounds or mixtures having similar chemical properties.

The term “control” refers to subjects or samples which provide a basis for comparison for experimental subjects or samples. For instance, the use of control subjects or samples permits determinations to be made regarding the efficacy of experimental procedures. In some embodiments, the term “control subject” refers to animals, which receive a mock treatment (e.g., saline).

The terms “patient” and “subject” refer to a mammal or an animal or a human that is a candidate for receiving medical treatment.

As used herein, the term “injured tissue” refers to a disruption of the normal continuity of structures caused by a physical (e.g., mechanical) force such as in incisions caused by surgery, a biological (e.g., thermic or actinic force), or a chemical means. In particular, the term “tissue injury” encompasses ischemia in various tissues, stroke in the brain, disappearance of secretory cells in endocrine glands, ischemia of muscles, severed neural exons and any disruption of normal structure and function of tissues. The term “injured tissue” also encompasses contused tissues, as well as incised, stab, lacerated, open, penetrating, puncture, injuries caused by ripping, scratching, pressure, and biting. The term further encompasses ulcerations (i.e., ulcers), preferably ulcers of the gastrointestinal track.

As used herein, the term “tissue repair and regeneration” refers to a regenerative process with the induction of an exact temporal and spatial healing program that encompasses but is not limited to the processes of granulation, neovascularization, fibroblast, endothelial and epithelial cell migration, extracellular matrix deposition, re-epithelialization, reappearance of the cells characteristic to the treated tissue prior to the injury and remodelling of the tissue.

The term “neovascularization” refers to the new growth of blood vessels with the result that the oxygen and nutrient supply is improved. Similarly, the term “angiogenesis” refers to the vascularization process involving the development of new capillary blood vessels.

The term “granulation” refers to the process whereby small, red, grain-like prominences form on a raw surface (that of injured tissues or ulcers) as healing agents.

The term “cell migration” refers to the movement of cells (e.g., fibroblast, macrophages, endothelial, epithelial, stem cells etc.) to the injured tissue.

The term “extracellular matrix” refers to the secretion by cells of fibrous elements (e.g., collagen, elastin, reticulin), link proteins (e.g., fibronectin, laminin), and space filling molecules (e.g., glycosaminoglycans). The extracellular matrix may contain additional components such as proteins and other molecules which may or may not be unique to each tissue

The term “re-epithelialization” refers to the reformation of epithelium over a denuded surface (e.g., injured tissue).

The term “regeneration” refers to the conversion of the injured tissue into a structurally and functionally healthy tissue similar that prior to the injury.

As used herein, the terms “localized” and “local” refer to the involvement of a limited area. Thus, in contrast to “systemic” treatment, in which the entire body is involved, usually through the vascular and/or lymph systems, localized treatment involves the treatment of a specific, limited area. Thus, in some embodiments, discrete injuries are treated locally using the methods and compositions of the present invention.

As used herein, the term “medical devices” includes any material or device that is used on, in, or through a patient's body in the course of medical treatment for a disease or injury. Medical devices include, but are not limited to, such items as medical implants, injured tissue care devices, drug delivery devices, and body cavity and personal protection devices. The medical implants include, but are not limited to, injections, urinary catheters, intravascular catheters, dialysis shunts, injured tissue drain tubes, skin sutures, vascular grafts, implantable meshes, intraocular devices, heart valves, collagen sheets, synthetic or natural matrices, and the like.

As used herein, “α-gal nanoparticles suspension” include, but are not limited to conventional suspensions of α-gal nanoparticles in a fluid aqueous vehicle such as, but not limited to, saline (physiological sodium chloride solutions), phosphate buffered saline, or any other fluid or gel. Suitable additives or auxiliary substances are isotonic solutions, such as physiological sodium chloride solutions or sodium alginate, demineralized water, stabilizers, collagen containing substances such as Zyderm II or matrix-forming substances such as povidone and collagen sheets. To generate a gel basis, formulations, such as aluminum hydroxide, polyacrylacid derivatives, collagen and cellulose derivatives (e.g., carboxymethyl cellulose), fibrin and plasma clots are suitable. These gels can be prepared as hydrogels on a water basis or as oleogels with low and high molecular weight paraffines or Vaseline and/or yellow or white wax. As emulsifier alkali soaps, metal soaps, amine soaps or partial fatty acid esters of sorbitants can be used, whereas lipids can be added as Vaseline, natural and synthetic waxes, fatty acids, mono-, di-, triglycerides, paraffin, natural oils or synthetic fats. The devices comprising α-gal epitopes and anti-Gal antibodies according to the invention can also, where appropriate, be administered topically and locally, in the region of the injured tissue, in the form of nanoparticle/antibody complexes, or complexes between any antigen and its corresponding antibody, or complement activating substances.

The term “semi-solid filler” refers to gel or gel-like materials that can include α-gal nanoparticles or α-gal carrying molecules and can fill various spaces in the body. The gel consistency of the semi-solid filler enables the diffusion in it of antibodies such as the anti-Gal antibody, complement and other proteins for the recruitment of macrophages into areas where the semi-solid filler is applied. Non-limiting examples for semi-solid fillers are plasma clot, hydrogel and fibrin glue.

The term “biomaterial” refers to any material that is used for implantation and is of synthetic source or natural source. It further includes tissues and organs from various species which may or may not undergo decellularization process and/or crosslinking treatment for the purpose of implantation into patients in order to induce repair and regeneration of injured or nonfunctioning tissues and organs.

The term “decellularization” refers to a process that involves immersion or perfusion of a tissue or organ in detergent solutions that solubilize cell membranes, remove cells and nuclei in order to produce a tissue or organ implant containing extracellular matrix without live cells.

### DETAILED DESCRIPTION OF THE INVENTION

### I. General

The present invention is related to the field of tissue regeneration and tissue engineering by various synthetic and natural biomaterials. In particular, the present invention provides compositions and methods for inducing rapid recruitment of macrophages into the injured tissues and into biomaterial implants and for activation of the recruited macrophage. This invention teaches how to recruit and activate macrophages by injection, or by other means of delivery, a preparation comprising synthetic nanoparticles presenting an α-gal epitope having a terminal α-galactosyl to an injured tissue of a subject having endogenous anti-Gal antibody, to produce a treated injured tissue. In some embodiments, the terminal α-gal is selected from the group consisting of Galα1-3Gal-R, Galα1-2Gal-R, Galα1-6Gal-R and Galα1-6Glc-R. The α-gal epitopes on the nanoparticles further include oligosaccharides available from Dextra Laboratories (Reading, U.K.), but are not limited to: i) Galα-3Gal glycolipids: α1-3 galactobiose (G203); linear B-2 trisaccharide Galα1-3Galβ1-4GlcNAc (GN334); and Galili pentasaccharide (L537). Various other glycoconjugates with α-gal epitopes available from Dextra Laboratories (Reading, U.K.) include for instance: Galα1-3Galβ1-4Glc-BSA (NGP0330); Galα1-3Galβ1-4(3-deoxyGlcNAc)-HAS (NGP2335); Galα1-3Galβ1-4GlcNAcβ1-HDPE (NGL0334); and Galα1-3Gal-BSA (NGP0203) all which may be linked to a lipid or to other materials that form nanoparticles. Another non-limiting example is the Elicityl (France) Galα1-3Gal (also called Galili) series of carbohydrate chains of various sizes carrying α-gal epitopes and having or lacking a linker all which may be linked to a lipid or to other materials that form nanoparticles. An additional non-limiting example is from Sigma-Aldrich “FSL-Galili” (F9432) also produced by KODE Biothech (Auckland, NZ). The synthetic carbohydrate chain may have one, two or several branches (also called antennae) carrying synthetic α-gal epitopes. Several non-limiting examples of additional macromolecules with α-gal epitopes that are suitable for injection and subsequent in situ binding to anti-Gal antibodies and local activation of complement include: mouse laminin with 50-70 α-gal epitopes (Galili, Springer Seminars in Immunopathology, 15:155, 1993), multiple synthetic α-gal epitopes linked to BSA (Stone et al., Transplantation, 83:201, 2007), GAS914 produced by Novartis (Zhong et al., Transplantation, 75:10, 2003), the α-gal polyethylene glycol conjugate TPC (Schirmer et al., Xenotransplantation, 11: 436, 2004), and α-gal epitope mimicking peptides linked to a macromolecule backbone (Sandrin et al., Glycocon J, 14: 97, 1997) and rabbit α-gal glycolipids from red cell membranes that are isolated according to size and used for α-gal nanoparticles preparation.

In some preferred embodiments, the α-gal epitope used for preparation of synthetic α-gal nanoparticles is part of a molecule selected from the group consisting of a glycolipid (e.g., α-gal epitopes on carbohydrate chain that is linked to ceramide), a glycoprotein (e.g., α-gal albumin), a proteoglycan, a glycopolymer (e.g., α-gal polyethylene glycol) and any other natural or synthetic spacer. In some particularly preferred embodiments, the glycolipid comprises α-gal nanoparticles in which the nanoparticles have on their surface α-gal epitopes that are capable of binding the anti-Gal antibody. Also provided are methods in which the preparation further comprises anti-Gal antibodies bound to the α-gal nanoparticles. In some preferred embodiments, the preparation is part of a tissue repair device selected from the group consisting of α-gal nanoparticles suspension, gels, semi-permeable films, ointments, foams synthetic biomaterials, natural biomaterials including decellularized tissues and decellularized organs such as, but not limited to, heart tissue liver organ, intestinal tissue and urinary bladder tissue or organ, all of which contain α-gal nanoparticles. In some embodiments the nanoparticles are of large size visible in a microscope or of submicroscopic size and may be referred to as α-gal liposomes.

In some embodiments, the glycolipid preparation comprising α-gal nanoparticles is derived from a source selected from the group consisting of rabbit red blood cells, bovine red blood cells, and other non-primate mammalian cells. In another embodiment the glycolipid preparation comprising synthetic α-gal nanoparticles. In addition, the present invention provides methods, comprising: providing; a subject having endogenous anti-Gal antibody and an injured tissue; and a preparation comprising an α-gal epitope having a terminal α-galactosyl; and applying the preparation to the injured tissue to produce a treated injured tissue. In some embodiments, the terminal α-galactosyl is selected from the group consisting of Galα1-3Gal, Galα1-6Gal and Galα1-2Gal. In some preferred embodiments, the α-gal epitope is part of a molecule selected from the group consisting of a glycolipid (e.g., α-gal epitope linked to ceramide), a glycoprotein (e.g., α-gal albumin), proteoglycan and a glycopolymer (e.g., α-gal polyethylene glycol). In some particularly preferred embodiments, the glycolipid comprises synthetic α-gal nanoparticles. Also provided are methods in which the preparation further comprises anti-Gal antibodies bound to the α-gal nanoparticles. In some preferred embodiments, the preparation is part of an injured tissue care device selected from the group consisting of biodegradable material such as collagen, alginate or cellulose, gels, biological matrices, semi-permeable films, and foams. In some embodiments, the glycolipid preparation is derived from a source selected from the group consisting of rabbit red blood cells, bovine red blood cells, and other non-primate mammalian cells. Also provided are methods in which the glycolipid preparation further comprises an antibiotic. Moreover, in some particularly preferred embodiments, the applying comprises one of the groups consisting of injection into the injured tissue, suspension in isotonic solution, ointment, lotion, topical solution, decellularized tissue from an animal and biodegradable patch, hydrocolloid and hydrogel.

In some preferred embodiments, the applying is under conditions such that complement activation within or adjacent to the injured tissue treated with synthetic α-gal nanoparticles is enhanced, in some embodiments, complement activation comprises production of C5a, C4a and/or C3a. In some preferred embodiments, the applying is under conditions such that one or more of the following take place: monocyte and macrophage recruitment within or adjacent to the injured tissue treated with synthetic α-gal nanoparticles is enhanced; injured tissue repair and regeneration are accelerated as a result of local monocytes and macrophage secretions; and angiogenesis in the injured tissue is enhanced. In some embodiments, the subject is selected from the group consisting of a human, an ape, an Old World primate, and a bird.

It is contemplated that the recruited and activated macrophages by the method described in this invention will induce repair and regeneration of the injured tissue or of the biomaterial implant by rapid and effective recruitment of stem cells by the activated macrophages or by the function of some of the recruited macrophages as stem cells. In some embodiments, the present invention provides treatments for tissue repair and regeneration in normal subjects and in subjects having tissues or organs with impaired biological activity, including but not limited to ischemic myocardium, injured nerves, injured cartilage including hyaline cartilage, elastic cartilage and fibrocartilage, injured skeletal muscle, injured smooth muscle, injured connective tissue, injured liver, injured endocrine glands injured eyes including cornea and lens, injured ears, injured gastrointestinal tract and injured bone, and in subjects implanted with biomaterials comprised of decellularized tissues or decellularized organs, tissues fixed by crosslinking or semisolid fillers and collagen sheets, all containing α-gal nanoparticles which interact in situ with the natural anti-Gal antibody.

In some embodiments, the glycolipid preparation is derived from a source selected from the group consisting of rabbit red blood cells, bovine red blood cells, and other mammalian cells. In some embodiment the glycolipids with α-gal epitopes comprise nanoparticles that may also comprise natural or synthetic lipids including but not limited to phospholipids, diglycerides and triglycerides. Such nanoparticles may also comprise cholesterol. Also provided are methods in which the glycolipid preparation further comprises an antibiotic or vitamins. Moreover, in some particularly preferred embodiments, the applying comprises one of the group consisting of applying of α-gal glycolipids comprised nanoparticles by injection into the injured tissue, or by any other application method known to those skilled in the art. In some preferred embodiments, the applying is under conditions such that complement activation within or adjacent to the injured tissue is enhanced, in some embodiments, the complement activation comprises production of C5a, C4a and C3a. In some preferred embodiments, the applying is under conditions such that one or more of the following take place: monocyte and macrophage recruitment and activation within or adjacent to the injured tissue is enhanced; angiogenesis in the injured tissue is enhanced; recruitment of stem cells into the injured tissue by said macrophages is enhanced; resulting in enhanced healing, repair and regeneration of said injured tissue. The stem cells recruited by the macrophages are either from areas of uninjured tissue near the injury site or from other areas in the body. It is contemplated that the enhanced, repair and regeneration occurs since the stem cells recruited and activated by the macrophages migrating into the treated injury, take instructive cues from differentiated cells located nearby, from the extracellular matrix (ECM) and from the microenvironment, to differentiate into cells with biological activities similar to those comprising the tissue prior to injury. It is contemplated that the macrophages recruited and activated following anti-Gal/α-gal epitopes interaction can further recruit and activate stem cells residing within the injured site prior to formation of fibrosis tissue in that site, thereby restoring the biological activity of the tissue and decreasing, or completely preventing scar formation.

In some embodiments, the treated subject is selected from the group consisting of a human, an ape, an Old World primate, and a bird. In some embodiments, the injured tissue may be any tissue including, but not limited to: brain, skeletal muscle, smooth muscle, myocardium, connective tissue, eyes, ears, limbs, pancreas and other endocrine glands, nerve, liver, gastrointestinal tissue and organs, bone, and cartilage. Furthermore, the present invention contemplates embodiments in which the nanoparticles comprise antigens which bind antibodies circulating in human blood, including but not limited to: α-gal epitope linked molecules binding the natural anti-Gal antibodies, rhamnose linked molecules binding natural anti-rhamnose antibodies, blood group A antigens binding anti-blood group A antibodies in subjects that have has a B or O blood type, blood group B antigens binding anti-blood group B in subjects that have A or O blood type, tetanus toxoid (TT) which bind anti-TT antibody. In additional embodiment, the present invention also provides nanoparticles comprising a preparation comprising an α-gal epitope having a terminal α-galactosyl. In some preferred embodiments, the terminal α-galactosyl is selected from the group consisting of Galα1-3Gal, Galα1-2Gal and Galα1-6Gal. In some particularly preferred embodiments, the α-gal epitope is part of a molecule selected from the group consisting of a glycolipid (e.g., α-gal epitope linked via a carbohydrate chain or any other linker to ceramide or to part of fatty acid), a glycoprotein (e.g., α-gal albumin) a glycopolymer (e.g., α-gal polyethylene glycol), a proteoglycan and to any hydrocarbon. In some embodiments, the glycolipid comprises α-gal nanoparticles. Also provided are devices in which the preparation further comprises anti-Gal antibodies bound to the α-gal nanoparticles also referred to as α-gal liposomes. In some preferred embodiments, the device is in the form of one of the group consisting of injectable suspension in an aqueous fluid, adhesive bands, gels, semi-permeable films, ointments, hydrocolloids gel, hydrogels and foams. In some embodiments, the glycolipid preparation is derived from a source selected from the group consisting of rabbit red blood cells, bovine red blood cells, and other non-primate mammalian cells, in some embodiment the glycolipids are produced in vitro by chemical or biological synthesis, in some preferred embodiments, the glycolipid preparation further comprises an antibiotic.

In some embodiments the natural or synthetic biodegradable nanoparticles present the sugar rhamnose linked to any spacer and interact with anti-rhamnose antibody that is present in humans (Chen et al. ACS Chemical Biology 6:185, 2011). The rhamnose nanoparticles and rhamnose liposomes are collectively referred to as rhamnose nanoparticles ranging in size from 1 nm to 500 mm. When applied to injuries or incorporated into biomaterials used as implants rhamnose nanoparticles and rhamnose liposomes interact with anti-rhamnose antibodies. This interaction results in activation of complement, generation of chemotactic complement cleavage peptides as C5a and C3a that induce rapid recruitment of macrophages. These recruited macrophages bind via their Fey receptors the Fc portion of the anti-rhamnose antibodies coating the nanoparticles and thus are activated to secrete cytokines/growth factors and induce repair and regeneration of the injured tissue and further induce recruitment of stem cells. These recruited stem cells receive cues from the microenvironment and from the ECM to differentiate into cells that restore the biological activity of the treated tissue, or of the implant containing the rhamnose presenting nanoparticles.

The mechanistic basis for recruitment and activation of macrophages by α-gal nanoparticles and the use of these nanoparticles in several clinical settings are described below as non-limiting examples for this invention. Similar recruitment and activation of macrophages for inducing repair and regeneration of internal injuries can be achieved by additional substances containing α-gal epitopes which interact with the natural anti-Gal antibody including, but not limited to glycolipids, glycoproteins, proteoglycans and glycopolymer such as α-gal polyethylene glycol.

### II. The Natural Anti-Gal Antibody, α-Gal Epitopes and α-Gal Nanoparticles

Anti-Gal is the most abundant natural antibody in all humans constituting ˜1% of circulating immunoglobulins (Galili et al. J Exp Med 160:1519, 1984). Anti-Gal binds specifically to a carbohydrate antigen called the α-gal epitope with the structure Galα1-3Galβ1-4GlcNAc-R (Galili et al. J Exp Med 162:573, 1985). This antibody is produced throughout life in response to continuous antigenic stimulation by bacteria of the normal gastrointestinal flora (Galili et al. Infect Immun 56:1730, 1988). Anti-Gal is naturally produced also in Old World monkeys (monkeys of Asia and Africa) and in apes, however, it is absent in other mammals (Galili et al. Proc. Natl Acad Sci USA 84:1369, 1987). In contrast, other mammalian species including nonprimate mammals (e.g. mice, rats, rabbits, dogs, pigs, etc.), as well as prosimians and New World monkeys (monkeys of South America), lack the anti-Gal antibody but all produce its ligand the α-gal epitope, by using a glycosylation enzyme called α1,3galactosyltransferae (α1,3GT) (Galili et al. Proc. Natl Acad Sci USA 84:1369, 1987; Galili et al. J Biol Chem 263:17755, 1988).

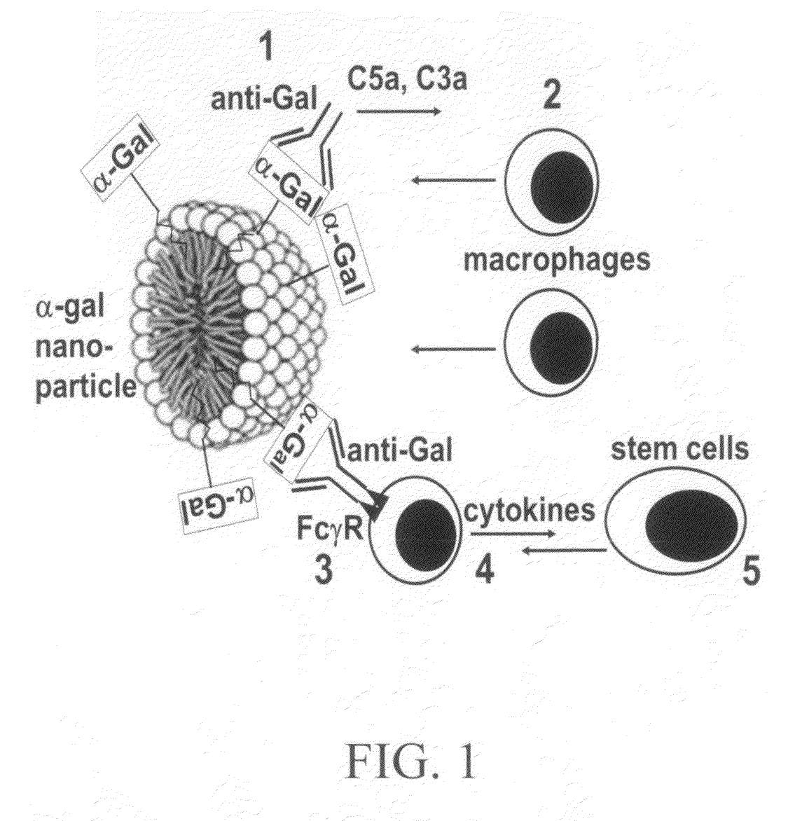

The activity of anti-Gal can be manipulated in humans by the use of α-gal nanoparticles which are schematically represented in FIG. 1. α-Gal nanoparticles can be prepared from various materials and they are characterized by presenting multiple α-gal epitopes. In a non-limiting example, α-gal nanoparticles are submicroscopic α-gal liposomes or micelles composed of glycolipids with multiple α-gal epitopes (α-gal glycolipids), phospholipids and cholesterol (Wigglesworth et al J Immunol 186:4422, 2011). Since α-gal glycolipids comprise most of the glycolipids in rabbit red blood cell (RBC) membranes and since these cell membranes are the richest source of α-gal glycolipids in mammals (Galili et al. supra 1987; Egge et al. J Biol Chem 260:4927, 1985, Galili et al. J Immunol 178:4676, 2007), rabbit RBC are a convenient natural source for preparation of α-gal nanoparticles (Wigglesworth et al. supra). For this purpose, glycolipids, phospholipids and cholesterol are extracted from rabbit RBC membranes in a mixture of chloroform and methanol (Galili et al. 2007 supra). The dried extract is sonicated in saline to generate liposomes (size of ˜1-10 μm) comprised of α-gal glycolipids, phospholipids and cholesterol and which present multiple α-gal epitopes of the glycolipids in the extract. These liposomes (referred to as α-gal liposomes) are further sonicated using a sonication probe into submicroscopic particles called α-gal nanoparticles which have the same composition as the α-gal liposomes, however their size range is 1-500 nm and preferably 10-300 nm. The α-gal nanoparticles suspension is further sterilized by filtration through a 0.2 μm filter. In this application the term α-gal nanoparticles also includes α-gal liposomes.

A schematic presentation of an α-gal nanoparticle is illustrated in FIG. 1. This nanoparticle has a wall of phospholipids in which α-gal glycolipids are anchored via the fatty acid tails of their ceramide portion. The illustrated glycolipid has 10 sugar units in its carbohydrate chain and 2 branches (antennae), each capped with an α-gal epitope. α-Gal glycolipids in rabbit RBC membranes are of various lengths ranging from 5 to 40 carbohydrate units carrying 1-8 branches each capped with an α-gal epitope (Galili et al. 2007 supra; Egge et al. 1985 supra; Hanfland et al. Carbohydrate Res 178:1, 1988; Honma et al. J Biochem (Tokyo) 90:1187, 1981).

Overall, the number of α-gal epitopes on α-gal nanoparticles is very high, corresponding to ˜10¹⁵ α-gal epitopes per mg α-gal nanoparticles (Wigglesworth et al. supra). From 1 liter of rabbit RBC it is possible to prepare 3-4 grams of α-gal nanoparticles. The α-nanoparticles are highly stable since they contain no tertiary structures. Accordingly, no changes in expression of α-gal epitopes were found in α-gal nanoparticles kept at 4° C. for 4 years in comparison with freshly produced α-gal nanoparticles.

The present invention teaches how to make α-gal nanoparticles in a synthetic form by the use of synthetic glycolipids such as but not limited to synthetic α-gal epitopes linked to a lipid via a carbohydrate chain and or without a linker. Such a synthetic glycolipids can be prepared by methods known to those skilled in the art. The phospholipid phosphatidyl choline or another lipid is dissolved in an organic solvent such as, but not limited to, methanol. A synthetic α-gal glycolipids, such as, but not limited to pentasaccharide linked to a diacyl lipid (FIG. 3) is dissolved together with the phosphatidyl choline in methanol at a molar ratio such as, but not limited to 1:10 α-gal glycolipid:phospholipid. The mixture is dried in a rotary evaporator, or in any other drying device as known to those skilled in the art. Subsequently, the dried mixture is sonicated in saline or other hydrophilic solutions to form synthetic α-gal nanoparticles comprised of phosphatidyl choline and synthetic α-gal glycolipid molecules. The synthetic α-gal nanoparticles can be in the form of liposomes comprised of at least one lipid bilayer or as a micelle in which the hydrophobic lipid chains are within the micelle facing each other and the hydrophilic part of the phospholipid as well as the carbohydrate chain of the glycolipid are facing the aqueous surrounding of the α-gal nanoparticle (FIG. 3). Synthetic α-gal nanoparticles may be prepared from any type of phospholipid and from synthetic glycolipids comprised of any kind of a lipid with one or more carbohydrate chains all or part of which carrying α-gal epitopes. The α-gal epitopes may be linked to the lipid by a spacer or directly by a carbohydrate chain. This linking is performed by methods known to those skilled in the art.

The synthetic α-gal epitopes such as the trisaccharides Galα1-3Galβ1-4Glc and Galα1-3Galα1-4GlcNAc, the disaccharide Galα1-3Gal and other synthetic oligosaccharides with terminal α-galactosyl units at the non-reducing end which may be obtained either as free oligosaccharide or linked by a linker to a lipid, protein, proteoglycan or any other molecule or polymer, all can be synthesized by chemical, biochemical, gene expression and/or enzymatic processes known to those skilled in the art. The human natural anti-Gal antibody binds to synthetic α-gal epitope trisaccharides, the disaccharide Galα1-3Gal and other synthetic oligosaccharides with terminal α-galactosyl units at the non-reducing end when they are used as free oligosaccharides or as oligosaccharides linked to molecules of various sizes. The affinity, i.e. strength of binding, of anti-Gal to such synthetic free oligosaccharides, was determined by equilibrium dialysis and found to be the highest when the synthetic oligosaccharide had the structure Galα1-3Galβ1-4GlcNAc, whereas the affinity of anti-Gal to synthetic Galα1-3Galβ1-4Glc is lower by approximately 30% than that to Galα1-3Galβ1-4GlcNAc (Galili and Matta Transplantation 62: 256, 1996). In addition, the affinity of anti-Gal to synthetic α-gal epitope trisaccharide Galα1-3Galβ1-4GlcNAc is 10 fold higher than the affinity of this antibody to the disaccharide Galα1-3Gal and 300 fold higher than the affinity to melibiose (Galα1-6Glc) or to methyl-galactoside (Galili and Mata 1996 supra). All these free carbohydrates were found to be capable of preventing by competition the binding of human natural anti-Gal antibody to the natural α-gal epitope expressed as approximately 50 epitopes/molecule on the mouse glycoprotein laminin and the same natural α-gal epitope expressed as 10⁷ epitopes/cell on pig endothelial cells (Galili and Mata 1996 supra). The human natural anti-Gal antibody was further shown to bind effectively and with high specificity to synthetic α-gal epitopes linked to silica beads. This was demonstrated by passing normal human serum on columns of various synthetic carbohydrate oligosaccharides linked to silica beads. Only beads with synthetic α-gal epitopes displayed binding of the natural anti-Gal (Galili U. Springer Seminars in Immunopathology 1993 supra). Anti-Gal eluted from the silica beads column with synthetic α-gal epitopes was further found to bind to natural α-gal epitopes on rabbit red cell membranes. All these observations imply that the human natural anti-Gal antibody binds to synthetic α-gal epitopes as well as to natural α-gal epitopes. These observations further imply that the natural anti-Gal antibody can bind effectively to synthetic α-gal nanoparticles and synthetic α-gal liposomes of various sizes presenting multiple synthetic α-gal epitopes.

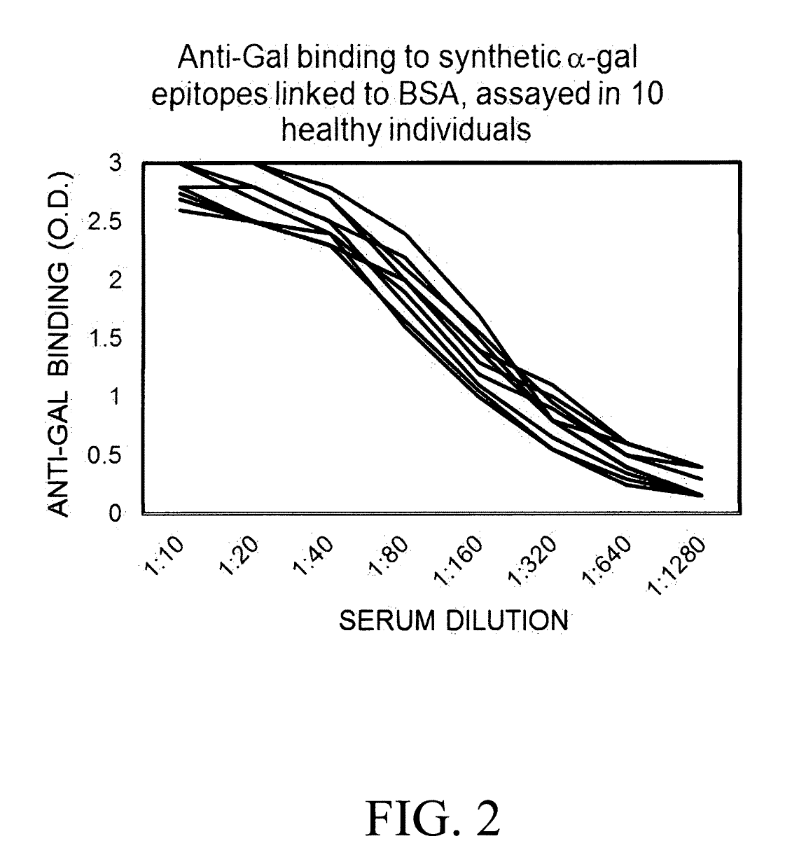

The extent of interaction of the natural anti-Gal antibody in sera of various individuals with synthetic α-gal epitopes is further demonstrated in FIG. 2 in ELISA assays in which synthetic α-gal epitopes linked to BSA (α-gal BSA produced by Dextra Laboratories, Reading, U.K.) served as solid-phase antigen coating the ELISA wells. FIG. 2 demonstrates anti-Gal binding to synthetic α-gal epitopes on α-gal BSA in sera of 10 healthy individuals. Binding to these synthetic α-gal epitopes yielding at least 1.0 O.D. could be demonstrated in all 10 serum samples even at serum dilutions of 1:160. Since all tested humans that are not severely immunocompromised produce the natural anti-Gal antibody which binds to synthetic α-gal epitopes in titers similar to those presented in FIG. 2, it is contemplated that application of synthetic α-gal nanoparticles and synthetic α-gal liposomes of any size to skin injuries will result in binding of the natural anti-Gal antibody to the synthetic α-gal epitopes on these α-gal nanoparticles and α-gal liposomes and initiate the sequential processes described in FIG. 1. It is further contemplated that administration into to humans with internal injuries said nanoparticles and liposomes to said injuries or use of implants containing said synthetic α-gal nanoparticles and synthetic α-gal liposomes will accelerate healing of said internal injuries and regeneration of said implants as a result of binding of the natural anti-Gal antibody to the synthetic α-gal epitopes on said α-gal nanoparticles and α-gal liposomes and initiate the sequential processes described in FIG. 1.

The studies on anti-Gal mediated acceleration of injury regeneration by α-gal nanoparticles cannot be performed in standard experimental animal models since animals such as mice, rats, guinea-pigs, rabbits and pigs, all produce α-gal epitopes on their cells and their extracellular matrix by the glycosylation enzyme α1,3galactosyltransferase (α1,3GT) and thus cannot produce the anti-Gal antibody (Galili et al 1987 supra; Galili et al. J Biol Chem 1988 supra). In addition to Old World monkeys, the only two nonprimate experimental animal models which are suitable for anti-Gal studies are α1,3GT knockout mice (GT-KO mice) produced in the mid-1990s (Thall et al. J Biol Chem 270:21437, 1995; Tearle et al. Transplantation 61:13, 1996) and α1,3GT knockout pigs (GT-KO pigs) produced in the last decade (Lai et al. Science 295:1089, 2002; Phelps et al. Science 299:41, 2003). These two knockout animal models lack α-gal epitopes and can produce anti-Gal. Old World monkeys, which naturally produce the anti-Gal antibody can serve as animal models, as well.

### III Anti-Gal/α-Gal Nanoparticles Interaction Induces Rapid and Extensive Macrophage Recruitment

Interaction between serum anti-Gal and α-gal epitopes on cells results in activation of the complement system. Transplantation of pig xenografts in monkeys is a demonstration of this complement activation. Binding of circulating anti-Gal antibody to the multiple α-gal epitopes on pig endothelial cells lining the blood vessels of pig kidney or heart xenografts, results in activation of the complement system that causes lysis of the endothelial cells, collapse of the vascular bed and hyperacute rejection of the xenograft within 30 minutes to several hours (Simon et al. Transplantation 56:346, 1998; Xu et al. Transplantation 65:172, 1998). A similar activation of complement occurs when serum anti-Gal binds to the multiple α-gal epitopes on α-gal nanoparticles. This complement activation results in the generation of chemotactic complement cleavage peptides that are among the most potent physiologic chemotactic factors for recruitment of macrophages. These include C5a and C3a complement cleavage peptides which induce rapid migration of macrophages into the site of α-gal nanoparticles application (FIG. 2) (Wigglesworth et al. supra).

In studies with α-gal nanoparticles injected intradermal in anti-Gal producing GT-KO mice, macrophages were found to be recruited by this chemotactic mechanism. The macrophages reached the injection site within 24 h and continued migrating into that site for several days (Wigglesworth et al. supra; Galili J. Immunol. Res. Vol. 2015, Article ID 589648). The identity of the migrating cells as macrophages could be determined by immunostaining with the macrophage specific antibody (Wigglesworth et al. supra). The macrophages were found at the injection site for 14-17 days and completely disappeared without changing skin architecture within 21 days. No granulomas and no detrimental inflammatory responses were found in such α-gal nanoparticles injection sites. It is contemplated that binding of the anti-Gal antibody to α-gal epitopes on synthetic α-gal nanoparticles described in FIG. 3 will result in a similar effects on macrophages as those with the α-gal nanoparticles described in Wigglesworth et al. (supra) since interaction of both types of nanoparticles present the same α-gal epitope which binds the same antibody, i.e. the natural anti-Gal antibody. It is further contemplated that injection of synthetic α-gal nanoparticles into the skin of GT-KO mice and subsequent binding of anti-Gal to the injected synthetic α-gal nanoparticles will result in rapid recruitment of macrophages similar to that demonstrated in Wigglesworth et al. supra and in Galili J. Immunol. Res. supra with α-gal nanoparticles generated from rabbit RBC, because of a similar activation of complement as that resulting in recruitment of macrophages.

### IV. Activation of Macrophages by Anti-Gal Coated α-Gal Nanoparticles

After the recruited macrophages reach the α-gal nanoparticles, the Fc “tails” of anti-Gal coating α-gal nanoparticles bind to Fcγ receptors (FcγR) on these macrophages (FIG. 1). This extensive binding to FcγR on macrophages was demonstrated by flow cytometry (Wigglesworth et al supra) and by scanning electron microscopy with anti-Gal coated α-gal nanoparticles that were incubated in vitro with cultured macrophages of α1,3GT knockout pig origin (GT-KO pig) (Galili, The Open Tissue Engineering and Regenerative Medicine Journal, 6: 1, 2013; Galili, J. Immunol. Res. Vol. 2015, Article ID 589648). Multiple α-gal nanoparticles attach to the macrophages via the Fc/FcγR interaction. In the absence of anti-Gal, no significant binding of α-gal nanoparticles to macrophages was observed. This Fc/FcγR interaction generates a trans-membrane signal that activates the macrophages to produce various cytokines and growth factors (referred to as cytokines/growth factors) that promote tissue repair and regeneration. Analysis of growth factors secretion into the culture medium demonstrated increased production of vascular endothelia growth factor (VEGF), whereas quantitative PCR demonstrated increased production of fibroblast growth factor (FGF), interleukin 1 (IL1), platelet derived growth factor (PDGF) and colony stimulating factor (CSF) (Wigglesworth et al. supra). It is contemplated that the Fc/FcγR interaction between the anti-Gal antibody coating α-gal nanoparticles and macrophages that was demonstrated by scanning electron microscopy (Galili, The Open Tissue Engineering and Regenerative Medicine Journal, supra; Galili, J. Immunol. Res. supra) and by flow cytometry (Wigglesworth et al. supra) will also between the Fc portion of anti-Gal coating synthetic α-gal nanoparticles and FcγR of macrophages recruited by these synthetic nanoparticles. It is further contemplated that this interaction between anti-Gal coating synthetic α-gal nanoparticles and macrophages will result in induction of cytokines and growth factors production by macrophages as that with the α-gal nanoparticles described in Wigglesworth et al. (supra) since interaction of both types of nanoparticles are antigen/antibody interactions between the same α-gal epitopes and the natural anti-Gal antibody.

### V. Macrophages Activated by Anti-Gal Coated α-Gal Nanoparticles Recruit Stem Cells

Among the cytokines secreted by the activated macrophages there are cytokines that induce recruitment of stem cells into the area of macrophage interaction with the α-gal nanoparticles. This can be inferred from studies with biologically inert polyvinyl alcohol (PVA) sponge discs (10 mm diameter and 3 mm thickness) which contain α-gal nanoparticles and are implanted subcutaneously in GT-KO mice producing anti-Gal. Cells which are recruited into PVA sponge discs containing α-gal nanoparticles, included, in addition to the migrating macrophages, also cells that display an extensive ability to proliferate (200-500 cells per colony formed from one cell within a period of 5 days). The frequency of these colony forming cells among cultured macrophages from PVA sponges is calculated to be 3-5 cells/10⁵ macrophages. This is a similar frequency as that of mesenchimal stem cells in the bone marrow (Eisenberg et al. Stem Cells. 24:1236, 2006). The ability to proliferate (i.e. self-renew) and form colonies is one of the main characteristics of stem cells. These findings indicate that the macrophages recruited by anti-Gal/α-gal nanoparticles interaction, are capable of further recruitment of cells with characteristics of stem cells in that the cells are capable of extensive proliferation.

In another example, the PVA sponge discs contained 150 μl suspension of 10 mg/ml α-gal nanoparticles mixed with 50 mg/ml homogenate of pig meniscus cartilage fragments devoid of α-gal epitopes. These PVA sponge discs were implanted subcutaneously in GT-KO mice, retrieved after five weeks, fixed, sectioned and stained with H&E or with trichrome (for staining collagen in blue). A section through a full size of the sponge disc demonstrated the formation of fibrocartilage comprised of relatively large areas of collagen fibers few chondrofibroblasts, similar to the structure of the meniscus fibrocartilage (Galili, Tissue Eng B 21: 231, 2015). In sponge discs containing meniscus fibrocartilage fragments but no α-gal nanoparticles, no fibrocartilage formation was detected. The de-novo formation of fibrocartilage demonstrated in Galili, Tissue Eng B supra, implies that the α-gal nanoparticles within the sponge discs recruited macrophages which were activated and secreted cytokines/growth factors that recruited stem cells, which, in turn, were directed by the fragmented meniscus cartilage to differentiate into fibrochondroblasts that produce collagen fibers characteristic to the meniscus cartilage. It is therefore contemplated that synthetic α-gal nanoparticles within implanted decellularized tissues or organs will similarly recruit macrophages that, in turn, will recruit stem cells into these implants. The recruited stem cells will be guided by the extracellular matrix (ECM) within the implant to differentiate into cells that regenerate the structure that characterized the implant prior to decellularization and restore the biological activity of that tissue. It is further contemplated that anti-Gal antibody will bind to synthetic α-gal nanoparticles similar to anti-Gal binding to α-gal nanoparticles described in Wigglesworth et al. (supra). Therefore, the recruitment of macrophages and subsequent recruitment of stem cells into PVA sponge discs implanted subcutaneously in GT-KO mice will be observed also in PVA sponge discs containing synthetic α-gal nanoparticles and implanted subcutaneously in GT-KO mice producing the anti-Gal antibody.

VI. Treatment of Injured Tissues by Administration of α-Gal Nanoparticles into the Injury Site

The U.S. Pat. No. 8,084,057, B2 “Compositions and methods for wound healing” describes the induction of wound healing in the skin by the application of α-gal liposomes to the wound area. The present invention teaches a method for treatment of internal injuries by injection of α-gal nanoparticles (α-gal liposomes, submicroscopic α-gal liposomes and micelles) into internal injuries for the purpose of recruitment of macrophages into the injury site. Alternatively, application of α-gal nanoparticles to injury sites may be performed by the use of semi-solid gels, plasma clot, fibrin glue, and other synthetic or natural biomaterials prepared to contain α-gal nanoparticles. The interaction of anti-Gal within the treated patient with the injected α-gal nanoparticles will activate the complement system and generate complement cleavage chemotactic peptides. These peptides will induce rapid and extensive migration of macrophages into the injection site within the injured tissue. The macrophages migrating into the injection site will further interact via their Fcγ receptor with the Fc portion of anti-Gal coating the α-gal nanoparticles as illustrated in FIG. 1. This interaction will activate the macrophage to secrete a variety of cytokines/growth factors which mediate angiogenesis and repair of the injured tissue. Among the secreted cytokines are those that recruit stem cells into the injury site. The recruited stem cells origin may be from the mesenchimal stem cells, stem cells from the uninjured adjacent tissue (referred to as progenitor cells) or from any other source in the body. It is contemplated that the invention described here for rapid recruitment and activation of macrophages by α-gal nanoparticles will ultimately induce repair and regeneration of the treated injury prior to the occurrence of the fibrosis process which is the default mechanism for healing of injured tissues in the body.

The submicroscopic α-gal nanoparticles may be prepared by sonication of α-gal liposomes into submicroscopic particles which can be sterilized by filtration through a filter that removes bacteria and protozoa as known to those skilled in the art. In one non-limiting example the filter can be with pores of 0.2 μm in size. The α-gal nanoparticles can be prepared also from any type of nanoparticles known to those skilled in the art and linking to these nanoparticles α-gal epitopes by a variety of chemical, biochemical and/or enzymatic methods known to those skilled in the art. The amount of α-gal nanoparticles which should be injected into injury sites may vary from 1.0 nanogram to 10 grams and preferably should be within the range of 1 mg and 100 mg.

In one embodiment α-gal nanoparticles may be injected into ischemic myocardium in order to induce rapid and extensive migration of macrophages into the injured tissue and the activation of the recruited macrophages within the ischemic myocardium. In post myocardial infarction, macrophages migrate to the injured myocardium, debride it of dead cells and secrete cytokines/growth factors that have various pro-healing effects, including, but not limited to angiogenesis and recruitment of stem cells. The recruited stem cells receive cues from the adjacent healthy cells, the microenvironment and the extracellular matrix (ECM) to differentiate into cardiomyocytes that regenerate the tissue and restore its physiologic activity (Minatoguchi et al. Circulation 109:2572, 2004; Dewald et al. Circ. Res. 96:881, 2005; Yano et al. J Am Coll Cardiol 47:626, 2006; Strauer et al. Circulation 106:1913, 2002). Myocardium with limited ischemic damage may display spontaneous regeneration by this mechanism. However in more extensive ischemic damage, migration of macrophages into injured myocardium and the recruitment of stem cells are processes that are too slow to prevent irreversible fibrosis which is the default mechanism for tissue repair. It is contemplated that this fibrosis may be reduced and possibly prevented by direct transendocardial injection of α-gal nanoparticles into the injured cardiac muscle by an injecting catheter, shortly after the ischemia event so that regeneration is enabled and fibrosis is prevented. The injection of α-gal nanoparticles into the ischemic myocardium may be performed also by any other method known to those skilled in the art. Injected α-gal nanoparticles will bind anti-Gal and induce rapid chemotactic migration of macrophages (Wigglesworth et al. supra; Galili et al. BURNS 36:239, 2010). The migrating macrophages recruited into the injection area within the ischemic myocardium will be activated and further recruit stem cells from the circulation or from uninjured adjacent myocardium. It is contemplated that the recruited stem cells may be guided by the microenvironment and by the ECM to differentiate into cardiomyocytes that repopulate the ischemic myocardium and restore its biological activity. It is contemplated that intramyocardial injection of synthetic α-gal nanoparticles will induce regeneration of ischemic myocardium similar to that induced by α-gal nanoparticles described above.

In another embodiment administration of α-gal nanoparticles will induce recruitment and activation of macrophages into nerve injury sites and thus may induce regeneration of nerves that are severed or damaged. Activated macrophages are pivotal in regeneration of injured nerves, as in spinal cord injury or in other nerve injuries in the body. Regeneration of nerves requires regrowth of multiple sprouts from the injured axons. These sprouts attempt to reconnect across the lesion and grow into the distal axonal tube of the damaged neurons. This axonal sprout growth depends on growth factors such as VEGF secreted by macrophages migrating to the injury site and inducing local angiogenesis (Dray et al. Proc Natl Acad Sci USA 106: 9459, 2009). If this growth of axonal sprouts is delayed because of insufficient recruitment and/or activation of macrophages in the injury site, the ongoing fibrosis will irreversibly prevent regeneration of the injured nerve. The α-gal nanoparticles applied to nerve injury site or injured brain tissue will bind anti-Gal and induce rapid macrophage migration and activation for the local secretion of VEGF. This process will result in local angiogenesis and growth of many axonal sprouts which increase the probability of axonal growth into the distal portion of the axonal tubes, ultimately inducing regeneration of the injured nerve (Galili, Immunology 140:1, 2013). The application of α-gal nanoparticles to induce nerve regeneration may performed by various methods known to those skilled in the art, including, but not limited to the use of a conduit in which the nanoparticles are mixed with a semi-solid filler such as keratin hydrogel-filled conduit (Horton, and Auguste Biomaterials 33:6313, 2012) or conduits containing nerve tissue ECM (Liu et al. Biomaterials 30:3865, 2009), or by using hydrogels, plasma clot, fibrin glue or any other type of α-gal nanoparticles suspension suitable for application to injured nerves. An example of a plasma clot containing α-gal nanoparticles as a semi-solid filler for application of α-gal nanoparticles was demonstrated in Galili, The Open Tissue Engineering and Regenerative Medicine Journal, supra. It is contemplated that administration of synthetic α-gal nanoparticles to injured nerves or brain tissue will induce regeneration of injured nerve similar to that induced by α-gal nanoparticles described above.

In yet another embodiment, α-gal nanoparticles may be applied to injured skeletal muscles or injured smooth muscles for the rapid recruitment and local activation of macrophages. A non-limiting example is the treatment of damaged skeletal muscle due to physical trauma. Injection of α-gal nanoparticles into the injured or damaged muscle tissue induces accelerated recruitment of macrophages into the injured muscle and activation of these macrophages by the interaction between the Fc portion of the anti-Gal antibody coating the α-gal nanoparticles and Fcγ receptors on the macrophages. It is contemplated that these activated macrophages secrete cytokines/growth factors that recruit myoblasts, which subsequently differentiate into functional myocytes that fuse into myotubes that comprise functional skeletal muscle fibers in treated skeletal muscle. It is contemplated that administration of synthetic α-gal nanoparticles into injured skeletal muscles will induce regeneration of injured muscle similar to that induced by α-gal nanoparticles described above.

In a further embodiment, application of α-gal nanoparticles into the synovial cavity, or into defects of damaged cartilage induces rapid and extensive recruitment of macrophages into these sites of damage and activation of these macrophages by the interaction between the Fc portion of the anti-Gal antibody coating the α-gal nanoparticles and Fcγ receptors on the macrophages. The recruited chondroblasts in turn secrete collagen and other cartilage matrix proteins and polysaccharides, resulting in repair and remodeling of the damaged cartilage. The introduced α-gal nanoparticles may be mixed with a homogenate of allogeneic cartilage or of xenogeneic cartilage devoid of α-gal epitopes which will function as extracellular matrix (ECM) that directs differentiation of recruited stem cells into chondroblasts secreting cartilage that repairs cartilage defects (Galili, Tissue Eng B supra). It is contemplated that administration of synthetic α-gal nanoparticles into cartilage defects without or with cartilage homogenate will induce regeneration of cartilage similar to that induced by α-gal nanoparticles described above.

In yet another embodiment, application of α-gal nanoparticles to bone injuries such as bone fractures induces rapid and extensive recruitment of macrophages into these sites of damage. In injured bones applied α-gal nanoparticles recruit and activate macrophages which secrete cytokines/growth factors that recruit cells becoming osteoclasts and osteoblasts which mediate repair and remodeling of the damaged bone. The introduced α-gal nanoparticles may be mixed with a homogenate of allogeneic bone or of xenogeneic bone devoid of α-gal epitopes which will function as extracellular matrix (ECM) that directs differentiation of recruited stem cells into osteoclasts and osteoblasts secreting bone material that remodels and repairs the injured bone tissue. It is contemplated that administration of synthetic α-gal nanoparticles which may or may not be mixed with homogenate of allogeneic or xenogeneic bone fragments devoid of α-gal epitopes into bone defects will induce regeneration of bone by recruitment of osteoblasts.

VII Incorporation of α-Gal Nanoparticles into Decellularized Tissues for Organ Regeneration

In the recent two decades there has been extensive research in the generation of biodegradable natural biomaterials which may be used in tissue engineering for tissue repair and regeneration (Atala, Curr Opin Biotechnol 20:575, 2009; Badylack: Lancet 379, 943, 2012). One exciting direction is the possible use of decellularized tissues and organs containing extracellular matrix (ECM) that maintains the original scaffold architecture and composition. Conservation of the ECM in whole organs is being achieved by advanced dynamic decellularization techniques using combination of various detergent and DNA destroying solutions (Atala, supra; Badylak, supra). The ECM in decellularized tissues and organs instructs stem cells to differentiate into cells that restore the biological function of the injured tissue. A number of studies have shown the use of scaffold derived from decellularized porcine bladder submucosa for urethral tissue engineering (Liu et al. supra), porcine decellularized myocardium for regeneration of the injured ventricular wall (Sarig et al. Tissue Eng Part A 18:2125, 2012) and porcine small intestinal submucosa for repair of small bowel tissue (Chen and Badylak, J Surg Res 99:352, 2001). Because of the decellularization processing, these biomaterials are porous (Crapo et al. Biomaterials 32:3233, 2011). Thus, soaking such biomaterials in an α-gal nanoparticles suspension will result in the nanoparticles being absorbed into these biomaterials. It is further possible that soaking dried biomaterials may increase α-gal nanoparticles penetration. When whole decellularized organs are used as biomaterials for tissue engineering, α-gal nanoparticles penetration can be achieved throughout the organ by perfusion with the α-gal nanoparticles suspension, similar to the perfusion of the decellularizing detergent solutions (Crapo et al. Supra). Following implantation, these nanoparticles will bind anti-Gal since the antibody is ubiquitous in the body. The resulting complement activation will induce rapid and extensive migration of the macrophages into the implant. Such migration was demonstrated in explanted GT-KO mouse hearts that were injected with α-gal nanoparticles and implanted subcutaneously into GT-KO mice producing the anti-Gal antibody. Although the heart implants were not connected to the recipients' circulation, within 2 weeks these implants contained large numbers of recruited macrophages, whereas heart implants injected with saline displayed only necrotic tissue with multiple neutrophils (Galili, The Open Tissue Engineering and Regenerative Medicine Journal, supra). Activation of these recruited macrophages by anti-Gal coated α-gal nanoparticles induces recruitment of stem cells which may be guided by the ECM to differentiate into the original cells of the decellularized tissue, similar to the differentiation of stem cells into fibrocartilage within PVA sponge discs demonstrated in Galili, Tissue Eng, supra). Thus, the α-gal nanoparticles within decellularized or non-decellularized implants can shift the dynamics of cell repopulation from fibroblasts infiltration and fibrosis as a default regenerative process, to the recruitment of stem cells that differentiate under the guidance of the ECM into the desired cells that restore the normal structure and biological activity of the organ. In the case of a heart patch this process may result in repopulation of the patch with cardiomyocytes that assist in the contraction of the ventricular myocardium, whereas in decellularized urinary bladder tissue this may result in the repopulation of the implant with smooth muscle cells and the covering of the surface with transitional epithelium (urothelium).

If the natural biomaterial is of a nonprimate mammalian origin, such as of porcine origin, the implant should be stripped enzymatically of autologous α-gal epitopes by α-galactosidase, or the implant should originate from α1,3galactosyltransferase knockout pigs (GT-KO pigs) devoid of α-gal epitopes. Since the α-gal epitope is present both on cells and on glycoproteins of the ECM (e.g. laminin), binding of anti-Gal to these epitopes on the ECM may result in immune mediated destruction of the ECM. Therefore, stripping the α-gal epitope from the ECM, or using tissues and organs that lack it will help preserving the ECM upon implantation in humans.

### VII Application of α-Gal Nanoparticles to Prevent Hair Loss and Baldness

Hair loss and baldness are initiated by a process of follicular miniaturization, in which the hair follicle begins to deteriorate. Hair follicles growth phase becomes shorter and eventually the hair follicles cease growing and becomes dormant, resulting in hair thinning due to hair loss, ultimately, leading to baldness. The experience on treatment skin wounds with α-gal nanoparticles demonstrated acceleration in proliferation of epidermis cells and thus in healing of wounds (Wigglesworth et al. supra). Since the hair follicles originate from epidermal cells and stem cells it is contemplated that application of α-gal nanoparticles or of synthetic α-gal nanoparticles to dormant hair follicles will activate these hair follicles and result in regrowth of hair. Since the skin epidermis seals the dormant hair follicles and prevents α-gal nanoparticles from reaching the hair follicles, application of the α-gal nanoparticles has to be in a way that enables them to reach near and in hair follicles. One non-limiting method for administration of the α-gal nanoparticles for this purpose is by partial removal of the epidermis by abrasion prior to application of the α-gal nanoparticles or synthetic α-gal nanoparticles to the bald or partly bald skin. Another non-limiting method for application of α-gal nanoparticles so they reach the area of the hair follicles is by multiple intradermal or subcutaneous injection of α-gal nanoparticles or synthetic α-gal nanoparticles. The injected α-gal nanoparticles or synthetic α-gal nanoparticles will recruit and activate macrophages. It is contemplated that the activated macrophages will secrete cytokines/growth factors that stimulate the hair follicle and recruit stem cells, ultimately resulting in stimulation of hair growth in the dormant hair follicles.

### EXPERIMENTAL

The following examples are provided in order to demonstrate and further illustrate certain preferred embodiments and aspects of the present invention and are not to be construed as limiting the scope thereof. The first set of examples (examples 1-5) demonstrates the rapid recruitment of macrophages by α-gal nanoparticles at various sites in the body. The second set of examples (examples 6-8) describes the recruitment of stem cells by macrophages that were first recruited and activated by α-gal nanoparticles.

In the experimental disclosure which follows, the following abbreviations apply: kDa (kilodalton); rec. (recombinant); N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); C (degrees Centigrade); α1,3GT (a 1,3 galactosyltransferase); GT-KO mouse (α1,3galactosyltransferase knockout mouse); GT-KO pig (α1,3galactosyltransferase knockout pig); BSA (bovine serum albumin); ELISA (enzyme linked immunosorbent assay); FcγR-Fcγ receptors; PBS (phosphate buffered saline); RBC (rabbit red blood cells);

### Example 1

### Recruitment of Macrophages by α-Gal Nanoparticles in Skin of GT-KO Mice

In vivo studies on the effect of α-gal nanoparticles were performed in α1,3 galactosyltransferase knockout (GT-KO) mice that were generated by Thall et al., (supra) and producing the anti-Gal antibody. The mice were induced to produce the anti-Gal antibody by pre-immunization with 50 mg pig kidney membranes. GT-KO mice producing anti-Gal antibody were injected subcutaneously with 10 mg α-gal nanoparticles in 0.1 ml saline. Other mice were injected subcutaneously with saline. Skin specimens from the injection site were obtained at different time points, fixed, stained with hematoxylin-eosin (H&E) and inspected under a light microscope. Multiple macrophages were seen in the area of injection within 24 h due the recruitment by chemotactic complement cleavage peptides generated as a result of anti-Gal/α-gal nanoparticles interaction ((Wigglesworth et al. supra; Galili, The Open Tissue Engineering and Regenerative Medicine Journal, supra; Galili, J. Immunol. Res. supra). The amount of migrating macrophages increased after 48 h. By day 6 the injection area is filled with macrophages that are very large. This morphology is likely to be the result of the activation by Fc/FcγR interaction with anti-Gal coated α-gal nanoparticles. The infiltrating macrophages are found in the injected skin even on day 14 post injection, but they disappear after 3 weeks. Subcutaneous injection of saline resulted in no recruitment of cells in the injection site at any time point. Overall the findings imply that α-gal nanoparticles injected into tissues rapidly recruit macrophages. This recruitment observed already within 24 h post injection of α-gal nanoparticles. This recruitment is temporary and lasts for 14-17 days. Subsequently the macrophages disappear from the injection site without affecting the architecture of the injection site and without causing any long-term granuloma of any detrimental inflammatory reaction. It is contemplated that synthetic α-gal nanoparticles will have the same effect on macrophage recruitment as that observed in Example 1.

### Example 2

### Interaction of Anti-Gal Coated α-Gal Nanoparticles With Fcγ Receptors on Macrophages

Recruited macrophages that reach the α-gal nanoparticles due to recruitment by complement cleavage chemotactic factors further interact via their Fcγ receptors (FcγR) with the Fc “tails” of anti-Gal coating α-gal nanoparticles (FIG. 1). This interaction results in generation of a trans-membrane signal that induces activation of the macrophages to produce and secrete a variety of cytokines and growth factors (referred to as cytokines/growth factors) which facilitate tissue repair and regeneration, including recruitment of stem cells. Measurements of the binding of anti-Gal coated α-gal nanoparticles to macrophages and the subsequent activation of these macrophages for the secretion of cytokines/growth factors were demonstrated in a study published in 2011 (Wigglesworth et al, supra) where increased production of vascular endothelial growth factor (VEGF), fibroblast growth factor (FGF), interleukin 1 (IL1), platelet derived growth factor (PDGF) and colony stimulating factor (CSF) were demonstrated. In the present example the actual binding of α-gal nanoparticles to macrophages was described in recent studies (Galili, The Open Tissue Engineering and Regenerative Medicine Journal, supra; Galili, J. Immunol. Res. supra) which showed by scanning electron microscopy that α-gal nanoparticles immunocomplexed (i.e. coated) with human natural anti-Gal antibody and incubated with cultured macrophages displayed extensive binding to macrophages via the Fc/FcγR interaction. The size of these α-gal nanoparticles is 10-300 nm. No binding of α-gal nanoparticles to macrophages was observed in the absence of natural anti-Gal antibody since in the absence of antibody on the nanoparticles, they cannot bind to the FcγR on the macrophage cell membrane. It is contemplated that synthetic α-gal nanoparticles coated with the natural anti-Gal antibody will display a similar Fc/FcγR interaction with macrophages as that observed in Example 2.

### Example 3

### Recruitment of Macrophages into a Heart Muscle Containing α-Gal Nanoparticles

This invention teaches how to induce macrophage recruitment into various organs for the purpose of repair and regeneration by the use of natural and synthetic α-gal nanoparticles. In one embodiment such recruitment of macrophages may be induced in ischemic myocardium following myocardial infarct in order to initiate a regeneration process of the injured myocardium and thus, to avoid fibrosis and scar formation. In another embodiment, inclusion of α-gal nanoparticles in decellularized organs or decellularized tissues implants will result in fast and effective recruitment of macrophages, which in turn will induce recruitment of stem cells. These recruited stem cells will be instructed by the ECM to differentiate into cells that restore the biological activity of the implant within the treated patients. The present example demonstrates the effective recruitment of macrophages into implants containing α-gal nanoparticles and the ability of the cytokines secreted from the recruited and activated macrophages to delay necrosis of the implanted ischemic tissue. Hearts were harvested from GT-KO mice and injected with 1 mg α-gal nanoparticles or with saline. Recipient GT-KO mice producing the anti-Gal antibody were implanted subcutaneously with the injected hearts without connecting any blood vessels to these hearts, i.e. the implanted hearts were ischemic. The implanted hearts were harvested after 2 or 4 weeks and subjected to histological analysis in order to determine whether the α-gal nanoparticles injected into these hearts can induce the recruitment of macrophages even if the organ is not connected to the blood circulation.

Implanted hearts harvested after 2 weeks that were injected with saline and thus contained no α-gal nanoparticles displayed eosinophilic staining of necrotic cardiomyocytes and multiple neutrophils in the tissue, but no infiltrating macrophages were observed. In contrast, implanted hearts that were injected with α-gal nanoparticles prior to implantation contained after 2 weeks multiple recruited macrophages despite the fact that the implants were not connected to the circulation of the recipients (Galili, The Open Tissue Engineering and Regenerative Medicine Journal, supra). All the nuclei in FIG. 8B are those of macrophages and not of cardiomyocytes. This is since nuclei of the ischemic cardiomyocytes are destroyed by endogenous nucleases. After 4 weeks, the recruited macrophages migrated into the adjacent non-injected myocardium. The activation of the recruited macrophages by anti-Gal coated α-gal nanoparticles resulted in secretion of cytokines that conserve the myocardium structure and prevent necrosis, as observed after 4 weeks both in areas adjacent to the recruited macrophages and in areas devoid of infiltrating macrophages (Galili, The Open Tissue Engineering and Regenerative Medicine Journal, supra). This preservation of structure of cardiomyocytes within hearts injected with α-gal nanoparticles occurred despite the absence of nuclei in these cells. Heart implants injected with saline completely disappeared during the period of 4 weeks post implantation.

Previous observations demonstrated the significance of macrophages in inducing angiogenesis and recruitment of stem cells in post infarct ischemic myocardium (Minatoguchi et al. supra; Dewald et al. supra; Yano et al. supra; Strauer et al. supra). In view of these studies, it is contemplated that the extensive migration and activation of macrophages following intramyocardial injection of α-gal nanoparticles may result in effective recruitment of stem cells which receive the appropriate cues from the conserved myocardium for differentiation into cardiomyocytes that restore myocardial function.

These observations with heart implants further imply that α-gal nanoparticles are very effective in inducing recruitment of macrophages into various injured tissues which are treated for induction of regeneration and are likely to mediate a similar effective recruitment of macrophages into decellularized organ and tissue implants, even if such implants are not connected to the circulation. It is contemplated that synthetic α-gal nanoparticles will display a similar recruitment of macrophages as that observed in Example 3.

### Example 4

### Recruitment of Macrophages into a Plasma Clot Containing α-Gal Nanoparticles

This example demonstrated the ability of a semi-solid filler containing α-gal nanoparticles to induce recruitment and activation of macrophages. Such semi-solid fillers are required for application of α-gal nanoparticles into spaces by injured tissues, while preventing extensive distribution of these nanoparticles in the body. The semi-solid filler in this example is in the form of a plasma clot gel containing α-gal nanoparticles. Plasma was mixed volume/volume with α-gal nanoparticles (10 mg/ml) and allowed to form a clot by adding calcium chloride (CaCl₂) at a final concentration of 10 mM. The CaCl₂ induces conversion of fibrinogen to fibrin and clot formation. These clots were placed on excisional wounds of anti-Gal producing GT-KO mice. Plasma clots were removed from the wounds 3 days and 6 days after the initiation of treatment. Macrophages recruited into the clot by anti-Gal/α-gal nanoparticle interaction and complement activation are clearly detected within 3 days after placing the plasma clot gel on the wound (Galili, The Open Tissue Engineering and Regenerative Medicine Journal, supra). The number of recruited macrophages greatly increases after 6 days when they seem to fill the plasma clot gel. These findings demonstrate the very effective mechanism of macrophage recruitment by complement cleavage chemotactic factors that are generated as a result of the antibody-antigen interaction between the anti-Gal antibody and α-gal nanoparticles. The activation of macrophages recruited into the plasma clot gel further resulted in secretion of cytokines/growth factors that induced rapid proliferation of the epidermis over this gel.

These findings imply that various semi-solid fillers or gels containing α-gal nanoparticles and enabling diffusion of various proteins, such as hydrogels, fibrin glue and plasma clots will enable the recruitment of macrophages into sites in the body where they are applied. This recruitment will be the result of anti-Gal/α-gal nanoparticles interaction and complement activation, thereby generating chemotactic complement cleavage peptides. The recruited macrophages migrating within these gels will be further activated by binding of anti-Gal coated α-gal nanoparticles to FcγR on macrophages and secretion of pro-healing cytokines/growth factors that help in regeneration of treated injured tissues. It is contemplated that synthetic α-gal nanoparticles will display a similar recruitment of macrophages into semi-solid fillers as that observed in Example 4.

### Example 5

### Recruitment of Stem Cells by α-Gal Nanoparticles into a Polyvinyl Alcohol (PVA) Sponge Discs

This example describes the ability of macrophages recruited and activated by α-gal nanoparticles to secrete cytokines/growth factors that can recruit stem cells. The analysis of stem cell recruitment was performed by subcutaneous implantation of PVA sponge discs containing 1 mg α-gal nanoparticles into in GT-KO mice producing anti-Gal. As indicated in a previous study (Galili et al. BURNS, supra), most cells recruited by α-gal liposomes into the PVA sponge discs for the period of 6 days post implantation are macrophages. The study was repeated with α-gal nanoparticles (i.e. α-gal liposomes of submicroscopic size). The recruited macrophages were retrieved from PVA sponge discs implanted for 6 days in anti-Gal producing GT-KO mice and plated in round cover-slip glasses in tissue culture wells. The cells were incubated on the cover-slip glasses for a period of 5 days. Subsequently the coverslips were washed and stained. The cells obtained from PVA sponge discs containing α-gal nanoparticles included cells that display an extensive ability to proliferate (200-500 cells per colony formed from one cell within a period of 5 days). The frequency of these colony forming cells among cultured macrophages from PVA sponges was found to be 3-5 cells/10⁵ macrophages. This is a similar frequency as that of mesenchimal stem cells in the bone marrow (Eisenberg et al. supra). Whereas macrophages migrating to the PVA lack the ability to proliferate, stem cells within the recruited populations are capable of proliferation and formation of cell colonies. These findings indicate that the macrophages recruited by anti-Gal/α-gal nanoparticles interaction, are capable of further recruitment of stem cells into the site of α-gal nanoparticle administration.

In a separate set of experiments the implantation of the PVA sponge discs was repeated in the GT-KO mice producing anti-Gal, however, these sponge discs were harvested after 5 weeks and subjected to histological staining and analysis. The PVA sponge discs contained a number of different types of tissues including nerves containing multiple axons, myotubes of striated skeletal muscle, connective tissue fibroblasts secreting collagen and blood vessels. The various types of tissues imply that the stem cells recruited by the activated macrophages into the PVA sponge disc include multipotential stem cells that can differentiate into various types of mature cells. It is contemplated that when such stem cells are recruited by macrophages that were recruited and activated by α-gal nanoparticles in injured tissues, the extracellular matrix (ECM) and the microenvironment within the implant guide the stem cells to differentiate into cells that restore the biological activity of the tissues in that implant. The recruitment of macrophages into an injured tissue is demonstrated in Example 3 above. Furthermore, a similar recruitment of stem cells by macrophages that were recruited and activated by α-gal nanoparticles can occur in implantation of decellularized tissue or organ containing α-gal nanoparticles. As implied in Galili, Tissue Eng B, supra, in such implants the recruited stem cells will be instructed by the ECM in the implant to differentiate into cells of the original type of the implant, thereby resulting in regeneration of the implanted tissues and organs. It is contemplated that synthetic α-gal nanoparticles will display a similar recruitment of stem cells.

### Example 6

### Accelerated Wound Healing by α-Gal Nanoparticles in a Pig Excisional Wound Model

The ability of α-gal nanoparticles to recruit and activate macrophages for subsequent recruitment of stem cells that induce tissue repair and regeneration was further demonstrated in the large animal model of GT-KO pigs. Similar to GT-KO mice, GT-KO pigs have a disrupted (knocked out) α1,3GT gene and thus, they lack α-gal epitopes and can produce the anti-Gal antibody (Phelps et al. supra; Yamada et al. Nature Med. 11:32, 2004; Galili Xenotransplantation, 20: 267, 2013). Full-thickness wounds (20×20 mm and ˜3 mm deep) on the back of 3 month old GT-KO pigs were covered with dressings coated with 100 mg α-gal nanoparticles or with saline. The borders of each wound were tattooed with 8 dots prior to treatment. Photographs were taken when wound dressings were changed every 3-4 days and wound surface area not covered by regenerating epidermis was measured (N=9) (Hurwitz et al. Plastic and Reconstructive Surgery, 129: 242e, 2012; Galili, J. Immuno. Res. supra).

On day 7, all wounds were filled with granulation tissue. The greatest gross morphology differences were observed on day 13. Part of the surface area in saline treated wounds was covered by regenerating epidermis due to physiologic healing processes. Thus, on average, the size of the non-covered wound area was ˜25 mm² (i.e. 0.5×0.5 cm). However, many of the wounds treated with 100 mg α-gal nanoparticles were completely covered by regenerating epidermis. On average, wounds treated with 100 mg α-gal nanoparticles had ˜90% smaller non-covered areas than saline treated wounds (Hurwitz et al. Supra). Full healing of saline treated wounds was observed on day 18-20 (not shown). Thus, healing time of treated wounds was shortened by 30-40%.

Wounds were harvested from pigs euthanized on days 7 and 13. Day 7 wounds treated with 100 mg α-gal nanoparticles had many more macrophages infiltrating the granulation tissue in the center of the wound compared with saline treated wounds. In the periphery of wounds, deposition of collagen by day 7 was more advanced in α-gal nanoparticle treated wounds than in saline treated wounds, as observed by trichrome staining. The increased collagen deposits imply earlier presence of collagen secreting fibroblasts in α-gal nanoparticle treated wounds.

Treatment of wounds with α-gal nanoparticles also resulted in extensive angiogenesis (Hurwitz et al. supra; Galili, J. Immuno. Res. supra). This could be demonstrated in day 13 wounds viewed in the area under the leading edge of the regenerating epidermis. Wounds treated with 100 mg α-gal nanoparticles displayed multiple blood vessels and even cisterna of blood. In contrast, the concentration of blood vessels was much lower in saline treated wounds, implying a much lower local secretion of VEGF in saline treated wounds than in α-gal nanoparticles treated wounds. The wound healing following treatment with α-gal nanoparticles did not prevent formation of hair shafts. These were detected in α-gal nanoparticles treated wounds already by day 13 (Hurwitz et al. supra). Thus, the healing of the injured skin treated with α-gal nanoparticles is faster than healing of saline treated wounds and it does not prevent formation of skin appendages. These observations imply that anti-Gal/α-gal nanoparticles interaction in pig wounds results in rapid recruitment and activation of macrophages that further recruit stem cells and induce angiogenesis and regeneration of the wound tissue into skin epidermis, dermis and skin appendages such as hair. In the treatment for regeneration of internally injured tissues, a similar sequence of events will result in the recruitment of macrophages followed by recruitment of stem cells into the areas in which α-gal nanoparticles are applied. It is contemplated that synthetic α-gal nanoparticles will display a similar accelerated wound healing as that observed in Example 6.

### Example 7

### Recruitment of Stem Cells by α-Gal Nanoparticles in the Presence of Meniscus Cartilage Fragments Results in Formation of Fibrocartilage

This example describes a process in which stem cells are recruited by macrophages which were recruited into PVA sponge discs. These stem cells are instructed by the ECM of a biomaterial comprised of dead tissue or decellularized tissue to differentiate into cells that regenerate the dead tissue. This process is illustrated by the formation of fibrocartilage which comprises meniscus cartilage as a result of recruitment of macrophages and stem cells by α-gal nanoparticles mixed with cartilage fragments. This process was studied in GT-KO mice implanted subcutaneously with polyvinyl alcohol (PVA) sponge discs containing this mixture a mixture of α-gal nanoparticles and small (10-100 μm) meniscus cartilage fragments from homogenized tissue (Galili, Tissue Eng B, supra). These PVA sponge discs had a diameter of 10 mm and are 3 mm thick. These sponge discs contained 150 μl suspension of 1 mg/ml α-gal nanoparticles mixed with 50 mg/ml homogenate of pig meniscus cartilage fragments devoid of α-gal epitopes. Removal of α-gal epitopes was achieved prior to fragmentation by 20 h incubation at 24° C. with 100 Units/ml recombinant α-galactosidase, followed by repeated washes (Stone et al. supra). The removal of α-gal epitopes from the cartilage fragments was necessary in order to prevent anti-Gal binding to these fragments. If such binding takes place (i.e. if the α-gal epitopes are not removed) the recruited macrophages will internalize the anti-Gal coated cartilage fragments. This process will prevent the formation of the appropriate microenvironment for the differentiation of stem cells into chondroblasts. The cartilage homogenate contained no live chondroblasts or chondrocytes.

PVA sponge discs containing α-gal nanoparticles and pig meniscus cartilage fragments devoid of α-gal epitopes were implanted under the skin, retrieved after five weeks, fixed, sectioned and stained with H&E or with trichrome (for staining collagen in blue) (Galili, Tissue Eng B, supra). Section through the sponge disc demonstrated areas of fibrocartilage formation. The generated fibrocartilage was similar in structure to meniscus cartilage which is characterized by relatively few fibrochondrocytes (identified by their elongated nuclei) and large amount of extracellular matrix comprised of the fibrocartilage matrix secreted by these cells. In sponge discs containing meniscus fibrocartilage fragments but no α-gal nanoparticles the de novo formation of fibrocartilage was residual. Most of the cells growing in the absence of α-gal nanoparticles formed fat tissue or loose connective tissue (Galili, Tissue Eng B, supra).

These observations imply that implantation of a decellularized organ or tissue containing the ECM scaffold and containing α-gal nanoparticles can result in recruitment of macrophages that will be activated and will recruit stem cells which will be guided by the ECM to differentiate into cells that restore the biological activity of the implanted organ or tissue. These observations further imply that application of α-gal nanoparticles mixed with cartilage homogenate in the form of semi-solid filler such as, but not limited to, hydrogel, fibrin glue or plasma clot gel into cartilage defect will recruit macrophages to that site and induce activation of the recruited macrophages. These activated macrophages will recruit stem cells that will differentiate into chondroblasts that will secrete their ECM including collagen and other cartilage matrix proteins and polysaccharides, resulting in repair and regeneration of the injured cartilage. Similarly, macrophages recruited and activated by the binding α-gal nanoparticles applied by semi-solid filler together with bone fragments or bone powder to damaged bone will secrete growth factors and cytokines that recruit osteoclasts and osteoblasts into the treated site for repair and regeneration of the damaged bone. It is contemplated that synthetic α-gal nanoparticles will induce a similar differentiation of stem cells in presence of cartilage ECM into regenerated cartilage tissue as that described in Example 7 and in Galili, Tissue Eng. B, supra. In the presence of articular cartilage fragments the differentiation of the recruited stem cells will be into hyaline cartilage. It is further contemplated that synthetic α-gal nanoparticles will induce a similar differentiation of stem cells in presence of bone ECM fragments into regenerated bone tissue.

### REFERENCES

Allison M R, Islam S. Attributes of adult stem cells. J. Pathol 217: 144, 2009
Atala A. Engineering organs. Curr Opin Biotechnol 20:575, 2009
Badylak S F, Weiss D J, Caplan A, Macchiarini P. Engineered whole organs and complex tissues. Lancet 379, 943, 2012
Bryant S M, Fukasawa M, Orita H, Rodgers K E, diZerega G S. Mediation of post-surgical wound healing by macrophages. Prog Clin Biol Res, 266:273, 1988
Chen M K, Badylak S F. Small bowel tissue engineering using small intestinal submucosa as a scaffold., J Surg Res 99:352, 2001
Chen W, Gu L, Zhang W, Motari E, Cai L, Styslinger T J, Wang P G. L-rhamnose antigen: a promising alternative to α-gal for cancer immunotherapies. ACS Chemical Biology 6:185, 2011
Clark R A. Basics of cutaneous wound repair. J Dermatol Surg Oncol, 19:693, 1993
Crapo P M, Gilbert T W, Badylak S F. An overview of tissue and whole organ decellularization processes. Biomaterials 32:3233, 2011
Dewald O, Zymek P, Winkelmann K, Koerting A, Ren G, Abou-Khamis T, Michael L H, Rollins B J, Entman M L, Frangogiannis N G. CCL2/Monocyte Chemoattractant Protein-1 regulates inflammatory responses critical to healing myocardial infarcts. Circ. Res. 96:881, 2005
DiPietro L A. Wound healing: the role of the macrophage and other immune cells. Shock 4:233, 1995
Dray C, Rougon G, Debarbieux F. Quantitative analysis by in vivo imaging of the dynamics of vascular and axonal networks in injured mouse spinal cord. Proc Natl Acad Sci USA 106: 9459, 2009
Egge H, Kordowicz M, Peter-Katalinić J, Hanfland P. Immunochemistry of I/i-active oligo- and polyglycosylceramides from rabbit erythrocyte membranes. Characterization of linear, di-, and triantennary neolactoglycosphingolipids. J Biol Chem 260:4927, 1985
Eisenberg C A, Burch J B, Eisenberg L M. Bone marrow cells transdifferentiate to cardiomyocytes when introduced into the embryonic heart. Stem Cells. 24:1236, 2006
Galili U. Evolution and pathophysiology of the human natural anti-alpha-galactosyl IgG (anti-Gal) antibody. Springer Seminars in Immunopathology, 15:155, 1993
Galili U. Anti-Gal: an abundant human natural antibody of multiple pathogeneses and clinical benefits. Immunology 140: 1, 2013
Galili. U. Anti-Gal Macrophage recruitment and activation by α-gal nanoparticles accelerate regeneration and can improve biomaterials efficacy in tissue engineering. The Open Tissue Engineering and Regenerative Medicine Journal, 6: 1, 2013 (available as open access online).
Galili, U. Acceleration of wound healing by α-gal nanoparticles interacting with the natural anti-Gal antibody. J. Immunol. Res. Vol. 2015, Article ID 589648 April 2015 (available as open access online).
Galili U. Avoiding detrimental human immune response against Mammalian extracellular matrix implants. Tissue Eng B 21: 231, 2015
Galili U, Rachmilewitz E A, Peleg A, Flechner I. A unique natural human IgG antibody with anti-alpha-galactosyl specificity. J Exp Med 160:1519, 1984
Galili U, Macher B A, Buehler J, Shohet S B. Human natural anti-alpha-galactosyl IgG. II. The specific recognition of alpha (1-3)-linked galactose residues. J Exp Med 162:573, 1985
Galili U, Clark M R, Shohet S B, Buehler J, Macher B A. Evolutionary relationship between the natural anti-Gal antibody and the Gal alpha 1-3Gal epitope in primates. Proc. Natl Acad Sci USA 84:1369, 1987
Galili U, Shohet S B, Kobrin E, Stults C L, Macher B A. Man, apes, and Old World monkeys differ from other mammals in the expression of alpha-galactosyl epitopes on nucleated cells. J Biol Chem 263:17755, 1988
Galili U, Mandrell R E, Hamadeh R M, Shohet S B, Griffiss J M. Interaction between human natural anti-alpha-galactosyl immunoglobulin G and bacteria of the human flora. Infect Immun 56:1730, 1988
Galili U, Wigglesworth K, Abdel-Motal U M. Intratumoral injection of alpha-gal glycolipids induces xenograft-like destruction and conversion of lesions into endogenous vaccines. J Immunol 178:4676, 2007
Galili U, Wigglesworth K, Abdel-Motal U M. Accelerated healing of skin burns by anti-Gal/alpha-gal liposomes interaction. BURNS 36:239, 2010
Galili U. α1,3Galactosyltransferase knockout pigs produce the natural anti-Gal antibody and simulate the evolutionary appearance of this antibody in primates. Xenotransplantation, 20: 267, 2013
Hanfland P, Kordowicz M, Peter-Katalinić J, Egge H, Dabrowski J, Dabrowski U. Structure elucidation of blood group B-like and I-active ceramide eicosa- and pentacosasaccharides from rabbit erythrocyte membranes by combined gas chromatography-mass spectrometry; electron-impact and fast-atom-bombardment mass spectrometry; and two-dimensional correlated, relayed-coherence transfer, and nuclear Overhauser effect 500-MHz 1H-n.m.r. spectroscopy. Carbohydrate Res 178:1,1988
Heinrich S A, Messingham K A, Gregory M S, Colantoni A, Ferreira A M, Dipietro L A, Kovacs E J. Elevated monocyte chemoattractant protein-1 levels following thermal injury precede monocyte recruitment to the wound site and are controlled, in part, by tumor necrosis factor-alpha. 11: 110, 2003
Honma K, Manabe H, Tomita M, Hamada A. Isolation and partial structural characterization of macroglycolipid from rabbit erythrocyte membranes. J Biochem (Tokyo) 90:1187, 1981 Horton R E, Auguste D T. Synergistic effects of hypoxia and extracellular matrix cues in cardiomyogenesis. Biomaterials 33:6313, 2012
Hurwitz Z M, Ignotz R, Lalikos J F, Galili U. Accelerated porcine wound healing after treatment with α-gal nanoparticles. Plastic and Reconstructive Surgery, 129: 242e, 2012
Knighton D R, Fiegel V D. The macrophages: effector cell wound repair. Prog Clin Biol Res, 299:217, 1989
Lai L, Kolber-Simonds D, Park K W, Cheong H T, Greenstein J L, Im G S, Samuel M, Bonk A, Rieke A, Day B N, Murphy C N, Carter D B, Hawley R J, Prather R S Production of alpha-1,3-galactosyltransferase knockout pigs by nuclear transfer cloning. Science 295:1089, 2002
Liu Y, Bharadwaj S, Lee S J, Atala A, Zhang Y. Optimization of a natural collagen scaffold to aid cell-matrix penetration for urologic tissue engineering. Biomaterials 30:3865, 2009
Lolmede K, Campana L, Vezzoli M, Bosurgi L, Tonlorenzi R, Clementi E, Bianchi M E, Cossu G, Manfredi A A, Brunelli S, Rovere-Querini P. Inflammatory and alternatively activated human macrophages attract vessel-associated stem cells, relying on separate HMGB1- and MMP-9-dependent pathways. J Leukoc Biol 85: 779, 2009
Low Q E, Drugea I A, Duffner L A, Quinn D G, Cook D N, Rollins B J, Kovacs E J, DiPietro L A. Wound healing in MIP-1alpha(−/−) and MCP-1(−/−) mice. Am J Pathol 159: 457, 2001
Minatoguchi S, Takemura G, Chen X H, Wang N, Uno Y, Koda M, Arai M, Misao Y, Lu C, Suzuki K, Goto K, Komada A, Takahashi T, Kosai K, Fujiwara T, Fujiwara H. Acceleration of the healing process and myocardial regeneration may be important as a mechanism of improvement of cardiac function and remodeling by postinfarction granulocyte colony-stimulating factor treatment. Circulation 109:2572, 2004
Phelps C J, Koike C, Vaught T D, Boone J, Wells K D, Chen S H, Ball S, Specht S M, Polejaeva I A, Monahan J A, Jobst P M, Sharma S B, Lamborn A E, Garst A S, Moore M, Demetris A J, Rudert W A, Bottino R, Bertera S, Trucco M, Starzl T E, Dai Y, Ayares D L. Production of α1,3-galactosyltransferase-deficient pigs. Science 299: 411, 2003.
Rappolee D A, Werb Z. Macrophage-derived growth factors. Curr Top Microbiol Immunol, 181:87, 1992
Sandrin M S, Vaughan H A, Xing P X, McKenzie I F Sandrin M S¹, Vaughan H A, Xing P X, McKenzie I F. Natural human anti-Gal alpha(1,3)Gal antibodies react with human mucin peptides. Glycocon J, 14: 97, 1997
Sang U, Au-Yeung G C, Wang Y, Bronshtein T, Dahan N, Boey F Y, Venkatraman S S, Machluf M. Thick acellular heart extracellular matrix with inherent vasculature: a potential platform for myocardial tissue regeneration. Tissue Eng Part A 18:2125, 2012
Schirmer J M, Fass D N, Byrne G W, Tazelaar H D, Logan J S, McGregor C G. Effective antiplatelet therapy does not prolong transgenic pig to baboon cardiac xenograft survival. Xenotransplantation, 11: 436, 2004
Seta N, Kuwana M. Human circulating monocytes as multipotential progenitors. Keio J. Med 56: 41, 2007
Shukaliak J A, Dorovini-Zis K. Expression of the beta-chemokines RANTES and MIP-1 beta by human brain microvessel endothelial cells in primary culture. J Neuropathol Exp Neurol 59: 339, 2000
Simon P M, Neethling F A, Taniguchi S, Goode P L, Zopf D, Hancock W W, Cooper D K. Intravenous infusion of Galalpha1-3Gal oligosaccharides in baboons delays hyperacute rejection of porcine heart xenografts. Transplantation 56:346, 1998
Snyderman R, Pike M C. Chemoattractant receptors on phagocytic cells. Annu Rev Immunol 2:257, 1984
Stappenbeck T S, Miyoshi H. The role of stromal stem cells in tissue regeneration and wound repair. Science, 324: 1666, 2009
Stein M, Keshav S. The versatility of macrophages. Clin Exp Allergy 22:19, 1992
Stone K R, Ayala G, Goldstein J, Hurst R, Walgenbach A, Galili U. Porcine cartilage transplants in the cynomolgus monkey. II I. Transplantation of alpha-galactosidase-treated porcine cartilage. Transplantation 65:1577, 1998
Strauer B E, Brehm M, Zeus T, Kostering M, Hernandez A, Sorg R V, Kogler G, Wernet P. Repair of infarcted myocardium by autologous intracoronary mononuclear bone marrow cell transplantation in humans. Circulation 106:1913, 2002
Tearle R G, Tange M J, Zannettino Z L, Katerelos M, Shinkel T A, Van Denderen B J, Lonie A J, Lyons I, Nottle M B, Cox T, Becker C, Peura A M, Wigley P L, Crawford R J, Robins A J, Pearse M J, d'Apice A J. The alpha-1,3-galactosyltransferase knockout mouse. Implications for xenotransplantation. Transplantation 61:13, 1996
Thall A D, Malý P, Lowe J B. Oocyte Gal alpha 1,3Gal epitopes implicated in sperm adhesion to the zona pellucida glycoprotein ZP3 are not required for fertilization in the mouse. J Biol Chem 270:21437, 1995
Wigglesworth K M, Racki W J, Mishra R, Szomolanyi-Tsuda E, Greiner D L, Galili U. Rapid recruitment and activation of macrophages by anti-Gal/α-Gal liposome interaction accelerates wound healing. J Immunol 186:4422, 2011
Xu Y, Lorf T, Sablinski T, Gianello P, Bailin M, Monroy R, Kozlowski T, Awwad M, Cooper D K, Sachs D H. Removal of anti-porcine natural antibodies from human and nonhuman primate plasma in vitro and in vivo by a Galalpha1-3Galbeta1-4betaGlc-X immunoaffinity column. Transplantation 65:172, 1998
Yano T, Miura T, Whittaker P, Mild T, Sakamoto J, Nakamura Y, Ichikawa Y, Ikeda Y, Kobayashi H, Ohori K, Shimamoto K. Macrophage colony-stimulating factor treatment after myocardial infarction attenuates left ventricular dysfunction by accelerating infarct repair. J Am Coll Cardiol 47:626, 2006
Yamada K, Yazawa K, Shimizu A, Iwanaga T, Hisashi Y, Nuhn M, O'Malley P, Nobori S, Vagefi P A, Patience C, Fishman J, Cooper D K, Hawley R J, Greenstein J, Schuurman H J, Awwad M, Sykes M, Sachs D H. et al. Marked prolongation of porcine renal xenograft survival in baboons through the use of alpha1,3-galactosyltransferase gene-knockout donors and the cotransplantation of vascularized thymic tissue. Nature Med. 11:32, 2004
Zhong R, Luo Y, Yang H, Garcia B, Ghanekar A, Luke P, Chakrabarti S, Lajoie G, Phillips M J, Katopodis A G, Duthaler R O, Cattral M, Wall W, Jevnikar A, Bailey M, Levy G A, Grant D R. Improvement in human decay accelerating factor transgenic porcine kidney xenograft rejection with intravenous administration of gas914, a polymeric form of alphaGAL. Transplantation, 75:10, 2003

I claim:

1. A method for inducing recruitment of macrophages into an injured internal or external tissue and for activation of said recruited macrophages to produce pro-healing cytokines and growth factors in a subject having endogenous anti-Gal antibody, comprising: administering to said injured internal or external tissue a composition that comprises synthetic α-gal nanoparticles, wherein:
i) said synthetic α-gal nanoparticles comprises α-gal epitope having a terminal α-galactosyl, and
ii) said composition is administered under conditions that increases an amount of macrophages in said injured internal or external tissue of said subject.

2. The method of claim 1, wherein said terminal α-galactosyl is selected from the group consisting of Galα1-3Gal, Galα1-2Gal, Galα1-6Gal and any α-galactose sugar units capable of binding anti-Gal antibodies.

3. The method of claim 1, wherein said α-gal epitope is free or part of a molecule selected from the group consisting of a natural or synthetic glycolipid, glycoprotein, and a glycopolymer.

4. The method of claim 1, wherein said synthetic α-gal nanoparticles are applied to induce regeneration of injured or atrophied internal or external tissues including: heart muscle, skeletal muscle, smooth muscle, connective tissue, ligament, cartilage, bone, nerve tissue, brain, spinal cord, liver, kidney, thyroid, parathyroid, pancreas, esophagus, stomach, small intestine, large intestine, blood vessels, lung, trachea, bronchi, bronchioles, eye, ear, skin and hair.

5. The method of claim 1, wherein said composition is part of an injury care device selected from the group consisting of injections, adhesive bands, compression bandages, gels, semi-permeable films, plasma clots, fibrin clots, water, solutions, suspensions, emulsions, creams, ointments, fibrin glue, aerosol sprays, collagen containing substances, stabilizers, sponges, drops, matrix-forming substances, extracellular matrix, foams or dried preparation.

6. The method of claim 1, wherein said administration occurs under conditions such that complement activation within or adjacent to said injured tissue is enhanced.

7. The method of claim 6, wherein said complement activation comprises production of complement fragments C5a, C4a and C3a.

8. The method of claim 1, wherein said applying is under conditions such that, polymorphonuclear cell, monocyte and macrophage recruitment within or adjacent to said injured tissue is enhanced.

9. The method of claim 1, wherein said applying is under conditions such that stem cell recruitment within or adjacent to said injury is enhanced.

10. The method of claim 1, wherein said applying is under conditions such that injury healing and tissue repair and regeneration is accelerated.

11. A method for inducing recruitment of macrophages into a biomaterial implant for activation of said macrophages to produce pro-healing cytokines and growth factors in a subject having endogenous anti-Gal antibody, comprising administering to said subject a biomaterial composition that comprises synthetic α-gal nanoparticles, wherein:
i) said α-gal nanoparticles comprises α-gal epitope having a terminal α-galactosyl and
ii) said composition is administered under conditions that increase the amount of macrophages in said injured internal tissue of said subject.

12. The method of claim 11, wherein said biomaterial is a natural decellularized tissue or decellularized organ selected from the group consisting of heart, urinary bladder, gall bladder, lung, trachea, bronchi, skeletal muscle, smooth muscle, connective tissue, ligament, cartilage, bone, nerve tissue, brain, spinal cord, liver, kidney, thyroid, parathyroid, pancreas, vagina, blood vessels, esophagus, stomach, small intestine, large intestine, blood vessels, eye, ear, and skin, or said biomaterial is a synthetic material.

13. The method of claim 11, wherein said biomaterial implant comprises collagen containing synthetic α-gal nanoparticles, cartilage fragments mixed with synthetic α-gal nanoparticles, or bone fragments mixed with synthetic α-gal nanoparticles.

14. The method of claim 11, wherein said biomaterial implant is dried or not dried and is immersed in a synthetic α-gal nanoparticle suspension to promote penetration of said synthetic α-gal nanoparticles into said biomaterial implant.

15. The method of claim 11, wherein said biomaterial implant is a biomaterial organ or tissue that is perfused with synthetic α-gal nanoparticles suspension in order to introduce said synthetic α-gal nanoparticles into said biomaterial organ or tissue.

16. The method of claim 11 in which anti-Gal antibodies are bound to said synthetic α-gal nanoparticles.

17. The method of claim 11, wherein said applying is under conditions such that complement activation within or adjacent to said biomaterial implant is enhanced.

18. The method of claim 17, wherein said complement activation comprises production of complement fragments C5a, C4a and C3a.

19. The method of claim 11, wherein the administration is under conditions such that macrophage recruitment within or adjacent to said biomaterial implant is enhanced.

20. The method of claim 11, wherein the administration is under conditions such that stem cell recruitment within or adjacent to said biomaterial implant is enhanced.
